SEQUENCE LISTING (11 sequences)

SEQ ID NO: 1 - DNA, 8247 bp, Escherichia coli
atcgcctcccattaactattgccagctacaagcaataattgtgccagtgttgattatccctgcggtgaataatgtcgatgatgtcgaaatgacacgtcgacacggcgacgaaattcatctttagcttaaaaatctctttaataacaataaattaaaagttggcacaaaaaatgcttaaagctggcatctctgttaaacgggtaacctgacaatgactatttgggaaataagcgagaaagccgattacatcgcacagcggcatcgtcgcctacaggaccagtggcacatctactgcaattcgctggttcaggggatcacgttatcgaaagcgcgcctgcatcacgccatgagctgcgcgccggacaaagaactctgtttcgtcctttttgaacattttcgcatttacgtcaccctggcggatggctttaacagccacaccatcgagtattacgtcgaaacaaaagatggcgaagacaaacagcggattgcgcaggcgcaactgagcattgacggcatgattgatggcaaggtcaacatccgcgatcgcgaacaggttctggaacactatctcgaaaaaatcgctggcgtttacgacagcttatacaccgctattgaaaacaatgtgccggtgaatttaagccaactggtaaagggacaaagcccggcagcatgagctgaggctttgcccgttttgcaggcgttacgcctgtttggggatgggcgtgtcgatgagtgtcgaaaatgacatttcatcggcatgttttcgtcaaaaatgacaatcacctgaggaatgcctggtgaatcgttttgtaattgctgactccacgctctgtatcggctgccacacttgtgaggccgcctgttcagagacgcatcgccagcacggcctgcaatcaatgccgcgcctgagagtgatgctgaatgaaaaagaatctgcgccgcagctctgtcaccactgtgaagatgcaccctgcgcggtggtctgcccggttaacgccatcacccgcgtcgatggggccgtgcagttgaatgaaagcctgtgcgtaagctgcaagctgtgcggcatcgcctgcccgtttggcgcaattgaattttccggcagccgtccgctggatattccggcaaacgccaataccccgaaagcgccaccggcaccgcctgctccggcgcgtgtcagcacattgcttgactgggtgccaggtattcgcgcgatcgccgtcaaatgtgacctttgtagctttgatgaacaaggtccggcctgcgcgcggatgtgcccgactaaagccctgcatctggtggataacaccgatatcgcccgcgtcagcaaacgtaagcgtgagctgacctttaacacggactttggcgatctcaccttgtttcagcaggctcaaagtggagaggctaaatgagcgcaatttccctgatcaatagcggcgtggcgtggtttgtcgccgccgctgttctggcatttctcttttcttttcaaaaagcgttaagtggctggatagctggaattggcggcgcggttggtagtctgtatacggcagccgcgggcttcactgtactgactggcgcggttggcgtgagcggtgcgctgtcgctggtaagctacgatgtgcaaatctctccgcttaacgcgatttggctgattacgctcggtctgtgcggtctgtttgtcagcctctacaacattgactggcatcgccacgcgcaggtgaagtgcaacggcttgcagatcaatatgttgatggctgccgccgtctgcgccgtcattgccagcaacctcggcatgttcgtggtaatggccgaaatcatggccctgtgcgcggtgttcctcaccagcaacagcaaagagggcaaactgtggtttgcgctggggcgtcttggcactctgctgctggcgattgcttgctggctgctgtggcagcgttacggcacgctggatctgcgcctgctggatatgcgtatgcaacagctgccgctcggttccgatatctggctgctcggagtgattggctttggcctgctggccgggattattccgctgcacggctgggtgccgcaggcacatgcgaacgcctctacaccagctgccgcgttgttttctacggtagtcatgaaaattggcctgctgggcattttaaccctgtcactgctgggcggtaatgcaccgctgtggtgggggatcgcgctgctggtgctcggcatgatcaccgcgtttgtcggtggtctgtatgcgctggtggagcacaacatccagcgcctgctggcttaccacaccctggaaaatatcggcatcatcctgctggggctgggcgctggcgtaacgggtatcgcgctcgaacaaccggcgctgattgctcttggcctggtcggtggtctgtaccatctgcttaaccatagcctgttcaaaagcgtactgttcctcggggcggggagcgtctggttccgtaccggtcatcgcgatatcgaaaaactcggtggtattggcaagaaaatgccggttatctccatcgccatgttagtcgggctgatggcaatggctgcgctgccgccgctgaatggttttgccggggaatgggttatctatcaatcatttttcaaactgagcaatagtggcgcgtttgttgcccgtctgctggggccgctgctcgctgtggggctggcaattaccggtgcgctggcggtgatgtgtatggcgaaagtctatggcgtcacgttcctcggcgcgccgcgcaccaaagaagccgaaaacgccacctgtgcgccgctcctgatgagcgtaagcgtagtggcactggcgatttgctgcgtaattggcggtgttgctgcgccgtggctactgccgatgctctctgctgctgtacctctgccgctggagcctgctaacaccaccgtttctcaaccgatgatcacgttgctgctgattgcctgcccgctgctgccattcatcattatggcgatttgcaaaggcgatcgtttgccatcgcgttcccgcggtgcggcctgggtgtgcggttacgaccacgaaaaatcaatggtgattaccgctcacggttttgccatgccggtgaaacaggcgtttgcgccggtgctgaaactacgcaaatggctgaatccggtgtctctggtgccgggctggcagtgcgaggggagtgcgttgctgttccgccggatggcgctggttgaactggcggtactggtggtgattattgtttcacgaggagcctgagaatgagtgttttatatccgttaattcaggcgctggtgttatttgccgttgcgccgctgctctccggtataacccgcgtggcgcgcgcccgcttgcataaccgtcgcgggccgggcgtgttgcaggagtatcgcgacattatcaaactgctggggcgtcagagcgtcggcccggatgcctccggctgggtgttccgcctgacgccgtatgtgatggtgggcgtcatgctgactatcgctactgcgctgccggtggtgaccgtcggttctccgctgccgcaactgggtgatttgatcaccttactgtatctctttgccatcgcgcgtttcttctttgccatttctggtctggataccggtagcccgtttaccgctatcggcgcgagccgtgaagcgatgcttggcgtgctggtcgaaccgatgctgctgcttggtctgtgggttgccgcacaggttgccggttccaccaacatcagcaacatcaccgacaccgtttatcactggccgctgagccagagcatcccgctggtactggcgctttgtgcctgtgcgttcgccacctttatcgaaatgggcaaactgccgttcgacctggcggaagccgagcaggagctgcaggaaggcccgctctctgaatacagcggcagcggctttggcgtcatgaaatggggtatcagcctgaaacagctggtggtgttgcagatgttcgtcggggtgtttattccgtggggacaaatggaaaccttcaccgccggtggactgctgctggcgctggtgattgccatcgtaaaactggtggtcggcgtcctggttatcgcgctgttcgaaaacagcatggcccgtctgcgtcttgatattactccgcgcattacctgggctgggtttggctttgcatttttagcgttcgtctccttgctggcggcgtgattaaagagagtttgagcatgtctgaagaaaaattaggtcaacattatctcgccgcgctgaatgaggcatttccgggcgtcgtgctggaccacgcctggcagaccaaagatcagctgactgtcaccgtaaaggtgaactacctgccggaagtggtggagtttctttactacaaacagggtggctggctgtcggtgctgtttggtaacgacgaacgcaaactgaatggtcattacgccgtttactacgtgctgtcgatggagaagggcactaagtgttggattacggttcgcgtcgaagttgacgccaacaaaccggaatatccgtccgtgacgccgcgcgttccggcggcggtgtggggcgagcgtgaagtgcgcgatatgtacggtttgattccggttggtctgccggatgaacgtcgtctggtgctgccggatgactggccggatgaactttatccgctgcgtaaagacagcatggattatcgtcagcgtccggcaccgaccaccgatgctgaaacctacgagttcatcaacgaactgggcgacaagaaaaacaacgtcgtgccgattggtccgctgcacgtcacttctgatgaaccgggccacttccgtctgttcgtcgatggcgaaaacattatcgacgccgactaccgtctgttctacgtccatcgcggcatggaaaaactggcggaaacccgtatgggttataacgaagtgaccttcctctctgaccgtgtgtgcgggatctgcggctttgcccacagcaccgcctacaccacgtcggtggaaaacgcgatgggtattcaggtgccagaacgtgcgcagatgatccgcgccattctgctggaggtagaacgcttgcactcgcatctgctcaaccttggcctggcctgtcactttaccggcttcgactccggctttatgcagttcttccgcgtgcgtgaaacctccatgaaaatggcagagatccttaccggtgcgcgtaaaacctacggcctgaacttgatcggcgggattcgtcgcgatctgctgaaagacgacatgatccagacccgccagctggcacaacagatgcgtcgtgaagtgcaggagctggtggatgtgctgctgagcactccgaacatggaacagcgcactgtcggcattggtcgtctggacccggaaatcgctcgcgacttcagtaacgtcggcccgatggtccgtgccagcggtcacgcccgtgatacccgcgccgatcacccgtttgtcggctatggcctgctgccaatggaagtccacagcgagcagggctgcgacgttatttcccgtctgaaagtgcgtatcaacgaagtctataccgcgctgaacatgatcgactacggtctggataacctgccgggtggcccactgatggtggaaggctttacctacattccgcaccgctttgcgctgggctttgccgaagcgccgcgcggcgatgatatccactggagcatgaccggcgacaaccagaagctgtaccgctggcgctgccgtgccgcgacctacgcgaactggccgaccctgcgctacatgctgcgcggcaacaccgtttccgatgcgccgctgattatcggtagcctcgacccttgctactcctgtaccgaccgcatgaccgtggtcgatgtgcgtaagaagaagagcaaagtggtgccgtacaaagaactcgagcgttacagcattgagcgtaaaaactcgccgctgaaataaggaatcgccatgtttacctttatcaaaaaagtcatcaaaaccggcacggcgacctcgtcttatccgctggagccgattgcggttgataaaaacttccgtggtaagccagagcagaacccgcagcagtgcatcggctgcgcggcctgcgtcaatgcctgcccgtcaaacgccttaacggttgaaactgacctcgccacaggagagcttgcctgggagtttaatcttgggcactgcatcttctgtggacgctgcgaagaagtctgcccgacggcggcgatcaaactgtcgcaagagtacgaactggcggtgtggaagaaagaagacttcctgcaacagtcccgcttcgcgctgtgcaactgccgcgtctgcaatcgtcctttcgccgtccagaaagagatcgactacgccattgcgctgcttaagcacaacggcgacagccgcgcggaaaaccaccgcgaaagctttgagacttgcccggaatgtaagcgccagaaatgcctggtgccgtccgaccgtattgaactgactcgccatatgaaagaggccatctgatgagcaatttattaggcccccgtgacgccaacggcattccggtccccatgacggtggatgaatccatcgccagcatgaaggcgtcgttactgaaaaaaatcaaacgttctgcctatgtttaccgcgtggactgcggcggctgcaacggttgcgaaatcgaaattttcggcacgctttcgccgctgtttgatgcagaacgcttcggcattaaagtcgttccttcaccgcgtcatgcggatattttactgtttaccggcgcggtcacccgtgcaatgcgatcccctgcgctgcgtgcgtggcagtccgcgccggacccgaaaatttgtatctcctacggtgcctgcggtaacagtggcgggatcttccacgatctctactgcgtgtggggcggtacggataaaattgtccctgtggatgtttatatccctggctgcccgccaacgcctgccgccacgctgtacggctttgcaatggcgctcggcctgctggagcagaaaattcacgcccgtgggccgggtgaactggatgaacaaccggcggagatcctgcatggtgatatggtgcagccgctgcgcgtgaaagtggatcgcgaagcacgtcgcctggcgggttatcgttacggtcgtcagattgccgatgattaccttacacagttagggcagggcgaagaacaggttgcacgctggctggaagcggaaaacgatccgcgtctgaacgagattgtcagccatctgaatcatgttgttgaagaggcgcgtatccgatgagtgaaaaggtggtgttcagtcaactgagccgtaaatttattgatgagaacgatgccacgcccgccgaggcgcagcaggtggtctattacagcctggcgattggtcaccaccttggggttatcgattgcctggaagcggcgctcacctgcccgtgggatgaatatctggcatggattgccactctggaggcaggcagtgaagcccgccgcaaaatggaaggcgtgccgaaatatggtgagatcgtcatcgacattaaccatgtgccgatgctggccaacgcattcgataaagcccgggcagcgcaaacttcgcagcagcaggaatggagtacaatgctgttaagtatgctgcatgatattcatcaggaaaacgccatctatttgatggtgaggagactgcgtgactgacgttttactctgtgttggcaatagcatgatgggcgatgatggcgcaggtccgctgctggcggaaaagtgcgccgccgcgccgaaaggtaactgggtggtgattgacggcggtagcgcaccggaaaacgacatcgtcgctatccgtgaactgcgcccgacacgactgctgattgtcgacgccacggatatggggctaaaccccggcgagatccgcatcatcgacccggatgatatcgccgagatgtttatgatgactacccataacatgccgttgaattaccttatcgaccagttgaaagaagatattggcgaagtgattttcctcggcattcagccggatatcgtcggcttttactacccgatgacccagccgattaaagatgcggtagaaaccgtttatcaacgactggaaggctgggaaggaaatggcggcttcgcgcagttagcggtggaagaagagtagtttttcattaaggaatcaggacagggatgttcttgatggggtgaaccagctctgatgccaaatgctaaattgcccgatgcgctgcgcttatcgggccttcatggttcgtgcgacatgtaggccggataaggcgttcacgccgcatccggcactgttacctactctaaatcttc

SEQ ID NO: 2 - DNA, 1964 bp, Desulfovibrio vulgaris
atgcccatgaccaactacggctggccatttccgtgacgcaaggggtgctgccccgcgcgctgggaccattcccggcgcgctggacgccttcgcgcgcggccaaggcgcctgacgccattccggtggcggcgggcgatgtcggaccggtacaaggcggcgcgagacgaggaggtgatcggacggcgtcgcgggtgccaacgacgtgatcgcaaccttcaaccacaggagacgtgctgatgagccgtatcgaaatggagaagatcttctacgaagaccacgctccggaccccaaggccgatcccgacaagctcttcttcatccagatcgatgaatcgaagtgcatcggatgcgacagctgccagcagtactgccccaccggcgccattttcggcgacacgggcgacgcgcacaagattccccacgaggaactgtgcatcaactgcggccagtgtctgacccactgccccgtgggcgccatctacgaatcgcagtcgtgggtgaccgagatcgagaagaagatcaaggccaaggacgtcaaggtcatcgccatgcccgcgcccgccgtgcgctacgccctgggcgacgctttcggcctgcccgtgggcaccgtgaccaccggcaagatgttctcggcgctcaaggaactgggcttcgaccactgctgggacaacgaattcaccgccgacgtgaccatctgggaagaaggcaccgaattcgtgcagcgcctgaccaagaagctggacaagccgctgccccagttcacctcgtgctgcccaggctggcacaagtacgtggaatcgctgtatccggaactattcccgcacatgtcgtcgtgcaagtcgcccatcggcatgctgggcacgctggccaagacctacggcgcggaccgcatgaagtacgaccgggccaaggtgtacacggtgtccatcatgccgtgcacagccaagaagtacgaaggcatgcgcccgcagttgtgggacagcggccacaaggacatcgacgccaccatcgacacccgcgaactggcgtacatgatcaagaaggccaagatcgacttcaccaagctgcccgacggcaagcgcgataccctgatgggcgaatccaccggcggcgcgaccctgttcggcgtcaccggcggcgtcatggaagcggcgctgcgctacgcctaccaggccgtcaccggcaagaagccggaaagcatggacttcaagggcgtgcgtggcttgcagggcgtgaaggaagccacggtgaacgtgggcggcgtggacgtgaaggtggccgtggtgcacggggccaggcgcttccatgacgtgtgcgaactggtgaaggcgggcaaggccccgtggcacttcatcgagttcatggcctgccccggcggttgcgtgtgcggcggcggccagccggtgatgcccggcgtgctcgaagccgccgaccgcaggtcgacccggatgtacgcgggcctcaagaagcgcctggccatggccagcgccagccgggcataggaggaatcaccatgcagatagtcaaccttacccggcgcggcttcctgaaggccgcctgcgtcgtcaccggcggcgccctgatcagcatccgcatgaccggcaaggccgtggccgccgccaagcagctgaaggattacatgatggaccgcatcaacggggtgtacggggcggacgccaagttcccggtgcgcgcctcgcaggataacgtgcaggtccagaagctgtacgcggacttcctggaaaagcccatgagccacaaggccgaacagttgctgcacacccactgggtggaccgctccaaggccatagaacgcatgaaggcgcaaggggcctaccccaacccgcgcgccaaggaattcgagggcaacacctacccctacgaatagctgcacggggccgcccaaacgccggaccttaggacgggtgcccagacgcgggcattgcccgccgtgcatgatccga

SEQ ID NO: 3 - DNA, 1800 bp, Desulfovibrio desulfuricans
gacggaggtagctatgaacttggttgaaatggaaaagattcagtatgtggatcagtcgcctgacccgcgagcgaatccggatgaactttttttcatccagattgatccggaaaaatgtatcggctgcgatacctgtcaggagtattgcccgacgggcgccatatttggcgataccgggagtgcgcatagtattcctcatgaagaaatctgcatcaattgcgggcagtgtcttactcattgccctgtcggagccatttacgaggtgcagtcctgggttcgggagctgagtgaaaaaatcaaagatccggagatcaaggtaatcgccatgccggctcctgctgtccggtatgggcttggagaatgtttcggcatgccggtgggcacggtaaccacgggaaagatgctgacagctttgcagatgctgggctttgaccacgtatgggacaatgagttcaccgcagatgtcaccatatgggaagaaggcaccgagtttgtcaaccgccttacagggcagattgacaagccgcttcctcagtttacctcttgctgccccggatggcacaaatatgtggaaagcttttatcctgagttgttcccgcatctttcaagctgcaaatctcccatcgggatgatgggcgcactggccaaaacgtatggaccggatgtgatgaaatatgatcggtccaaggtttatacggtttccattatgccctgtaccgctaaaaagtatgagggcatgcgcgcggacctgtggtccagcggctacaaagacattgacgcaaccatagacacccgcgaactggcctacatgataaaaaaggcaggcattgatttcgccgctctgccggacggcaaacgggacacgttgatgggtgattccaccggtggggccacgattttcggtgtatccggcggtgttatggaagcggctttgcgttatgcctacgaagctgtgaccggcaaaaaaccgtcgagctgggattttacgatggttcgcggcctgaacggtatcaaagaaggtactgtcaccattggcgatgcaaagataaacgtggcggtggttcacggagcaaagcgttttgcggaagtatgcgaggtgattaagacagggaaaagcccgtggcatttcatcgagtttatggcctgccccggaggatgtgtctgcggtggcggtcagcctgtgatgcccggagtactggaagctatggaccgcaaagtttccagaacctttgccggactcaaagagcgtctgaacagaatgtcgtccagcaaggcataggaggatgaaatgagtattgctgcattcacccgtcgtcagtttctgaaaggcggctgcatggcctgcggcgctgcaattgttggtatccgctttaccggaaaggcattagcagcggtcaagcaggttaaagagtatatgaccgaccgcataaacagtgtctacgctgcggacaaaacgtttgtgcatcgggcatcgcaggacaacccgcaggtaatcaaactgtacaaggactggcttgaacatcctatgagccataaggctgagcagtttctgcacatgcactggacagaccggtctcagggcatacagaaactgattgcggaacgccagtatcccaacccccgcgccggagaattcggcaacgggtatccttacgaataaccgctgaatcctccgacgacctgaaagaaggccttatctgactcaagggtcgtcttcgtttcctgcctcttttgcgccgcgctggtgccggtcggcaccggaatgtcactgcaggcgtcttccgcatgcgtggtttccgt

SEQ ID NO: 4 - DNA, 3180 bp, Desulfovibrio fructosovorans
cgtgctgcatacgatttctttattcatatcctttacttccccggcctcatcaccccggtaacacccttgaatcacgtgaaaaatttgccaaaccccattgacgttcaaacaaactcgtgattaactgcaaaagggaaatcgaacccggcgacgcaggaacgcacgcgtccgtcacatagaggcggcggcggccggttcgcctgcgacggccgcgtgccgaacgggtcaacggatgctacgtggcctggggatcatgggacgccccggaccatgcctctcgtaaaacaaaggaggacgtttatgaacttttccgtgggtcttggcaggatgaatgcggaaaaacggcttgtgcaaaacggcgtctcccgccgcgacttcatgaaattttgcgccaccgtggccgcggccatgggcatgggcccggcgttcgcgcccaaggtcgccgaagcattgacggccaaacaccgtccgtcggtggtctggctgcacaacgccgagtgcaccggctgcaccgaagcggcgatccggacgatcaaaccttatatagacgcgctcattctcgacaccatctccctggattaccaggagaccatcatggccgcggccggcgagacgtccgaggcggccctgcaccaggccctcgaaggcaaggacggctactacctcgtggtcgagggcggcctgcccaccatcgacggcggccagtggggcatggttgccggccatcccatgatcgagaccaccaagaaggccgcggccaaggccaagggcatcatctgcatccggcacctgcctcacggcggcgtccagaaggccaaacccaatcccagccaggccaagggcgtgtccgaagccctcggcgtcaagaccatcaacatccccggctgcccgcccaaccccatcaacttcgtgggcgccgtggtccatgtcctgaccaagggcatcccggatctcgacgagaacggccgtccgaagctcttctacggcgagctggtccacgacaactgtccgcgcctgccccacttcgaggcctccgaattcgcgccctccttcgattccgaagaggccaagaaaggcttctgcctctacgaactcggctgcaagggccccgttacctacaacaactgccccaaggtgctgttcaaccaggtcaactggcccgtccaggccggccacccctgcctcggctgcagcgagccggacttctgggacaccatgacgccgttctacgagcagggctaaccccctcctttgtagcggccagtacccggacaccttcaacagcaccgaacgtctcgtgacggaggaagcatatggctgagagcaaacccacgccacaatccaccttcaccggccccatcgtggtcgaccccattacccggatcgaaggtcacttgcggatcatggtcgaggtggaaaacggtaaggtcaaggacgcctggagctcctcgcagctcttccgcggcctggaaatcatcctcaaaggccgcgatccccgcgacgcccagcacttcacccagcgcgcctgcggcgtgtgcacgtacgtccacgccctggcctccagccgctgcgtcgatgacgccgtcaaggtcagcatcccggccaacgcccgcatgatgcgcaacctggtcatggcctcccagtatctccatgaccacctcgtccacttctatcacctgcacgccctcgactgggtcgacgtgaccgccgccctcaaggccgatcccaacaaggccgccaaactggcagcctccatcgacacggctcggaccggcaactcggaaaaggccctcaaggcagtccaggacaagctgaaagccttcgtcgagtccggacagctcggcatcttcaccaacgcctacttcctcggcggccacaaagcctactacctgccgcccgaggtcaacctcatcgccaccgcccactacctggaagccctgcacatgcaggtcaaggcggccagcgccatggccatcctcggcggcaagaacccccacacccagttcaccgtcgtgggcggctgctccaactaccagggcctgaccaaggacccgctggccaactacctggccctgagcaaggaagtctgccagttcgtcaacgagtgctacatccctgacctgctggccgtggccggcttctacaaggactggggcggcatcggcggcaccagcaactacctggccttcggcgagttcgccaccgacgacagctcccccgagaaacacctggccacctcgcagtttccgtccggcgtcatcaccggccgcgacctcggcaaggtggataacgtggacctcggcgccatctacgaagacgtcaagtactcctggtacgcccccggcggcgacggcaagcacccctacgactgcgtcaccgatcccaagtacaccaagctcgacgacaaggaccactactcctggatgaaggccccccgctacaagggcaaggccatggaagtcggtcccttggcccgcaccttcatcgcctacgccaaggggcagcccgacttcaaaaaggtcgtggacatggtcctcggcaaactctccgtcccggccacggccctgcattcgaccctcggacgcaccgccgcccgcggcatcgagaccgccatcgtctgcgccaacatggagaagtggatcaaggaaatggccgacagcggcgccaaggacaacaccctgtgcgccaagtgggagatgcccgaggagtccaagggcgtcggcctggccgatgctccccgcggctccctgtcccactggatccgcatcaagggcaagaagatcgacaacttccagctggttgtcccctcgacctggaacctcggtccccgggggccccagggcgacaagagcccggtggaagaggcccttatcggcacgcccatcgccgatcccaaacgcccggtcgaaatcctgcgcacggtccacgccttcgacccctgcatcgcctgcggcgtgcacgtcatcgagcccgagaccaacgagatcctcaagttcaaggtttgctaagagcggcaaccctctaatacgaaggacccgcttcgggccgggttcttcgtttctcaaggaggtcgcatgtccgataccccgcgcaaaatcctcatcctcggcctcggcaacatcctctacaccgacgagggcgtcggcgtgcgggccgtggagcgccttctcgaaacccacg

SEQ ID NO: 5 - DNA, 2756 bp, Desulfovibrio gigas
gtgttacattggacgtggcaaggatccaggtcgaggaatcgcctggagcgccggcgtctccgccgcgacttcatgaagttctcgcgcgccgtggccgtggccgccatgggtatgggtcccgcctttgcccccaaggttgccgaggcgttgaccgccaagaagcgccccagcgtggtgtatctgcacaatgcagaatgcaccggctgcagcgaatctgtgctgcggaccgttgacccgtacgttgacgaactcattcttgatgtgatttccatggattatcacgaaaccctcatggctggcgccggtcatgccgtggaagaagccctgcacgaagccatcaagggcgatttcgtctgcgtcattgaaggcggcattcccatgggcgacggcggctactggggcaaggtcggcggccgcaacatgtacgacatctgcgccgaagtggctcccaaggccaagggtggcgtggccatcggcaccgcgccacctacggtggccaagggccttggcccaaaggccaagcccaaccacgggcaccgtgggtgtgaacgaagccctgggcaaactgggcgtgaaggctatcaacatcgccggctgcccgccgaaccccatgaactttgtgggcaccgtggtgcatctgctcaccaagggcatgcccgagctggacaagcaaggccgcccggtgatgttcttcggcgaaaccgtgcacgacaactgcccccgtctgaagcacttcgaagcgggcgagtttgccacctcttcggctcccctgaagccaagaagggctactgcctctatgagctgggttgcaagggtcctgatacgtacaacaactgccccaagcagctcttcaaccaggtcaactggccggtccaggccgggcacccctgcatcgcctgcagcgagcccaatttctgggatctctattcgccgttctacagcgcctagacgacctgcggccaagctcatcgagcactagccaggaacttcgtttcgcacaggagaagacccatgtctgaaatgcaaggcaacaagatcgtcgtcgatcccatcacccggatcgagggccatcttcgcattgaagtggaagtggaaggcggcaagatcaagaacgcctggagcatgtccacgctgttccgcggcctggaaatgatcctcaagggccgcgacccgcgcgacgcccagcacttcacccagcgcgcctgggctgtgcacctatgtgcatgccctggcctccgtccgcgccgtggcaactgcgtgggcgtgaagattcccgaaaacgccactctcatgcgcaacctcaccatgggcgcgcaatacatgcacgaccacttggtgcacttctaccacctgcatgcccttgactgggtgaacgtggccaacgccctgaacgccgatccggcaaggctgcccgcctggccaacgatctctccccccgcaaaaccaccacggaaagcctcaaggccgtgcaggaaggtcaaggccctggtggaaagcggccagtgggcatcttcaccaacgcctacttcctgggcggccaccccgctatgtgctgccggcggaagtggacctcatcgccaccgcccactacctggaagccctgcgcgtgcaggtgaaggccgcccgggcatggcatcttcggcgccaagaacccccacacccagttcaccgtggtgggtggttgcaccaactacgactccctgcgtcccgaacgcatcgccgaattccgcaagctgtacaaggaagtccgcgagttcatcgagcaggtgtacatcacggacctgctggccgtggcgggttctacaagaactgggccggcatcggaaaacgtccaacttcctcacctgcggcgaattccccaccgacgaatacgatctgaactcccgctacaccccgcagcgtgatctggggcaacgacctcagcaaggtggacgacttcaaccccgacctcatcgaggagcacgtcaagtactcctggtacgaaggcgcggacgccaccatccgtacaagggcgtcaccaagcccaagtggaccgaattccacggcgaagatcgctactcctggatgaaggccccccggtacaagggcgaagtcgaagtggggccgttggtcccgtgctcgtggcttacgcaagaagcacgaacccaccgtcaaggctgtggatcttgtcctcaagaccctgggcgtgggcccgaagccctgttctccaccctgggccgcaccgccgcccgcgattcagtgcctcaccgccgctcaggaagtggaagtgtggctggacaagctggaagccaacgtcaaggctggcaaggacgatctctacaccgattggcagtaccccaccgagtcccagggcgtgggctttgttacgcgccccggcatgctctcccactggattgtccaacgtggcgcaagatcgagaacttccagcatgtggtgccctccacctggaacctcggcccccgctgcgccgaaaaactctccgctgtggaacaggccctcatcggtacgcccatcgccgatcccaagcgtcctgtggaaatcctgcgcaccgtgcactcctacgacccctgcatcgcctgcgctgcacgtgatcgatccggagtccaaccaggtgcacaagttccgcatcctgtagttccggacgccagacccaacgcaacaaccaggagcccggccttggcccggatcgtctaatcaaggtgttcatgtccaacgactctccccgcatcctcgtcatgggcgtcggcaacatcctg

SEQ ID NO: 6 - DNA, 2589 bp, Desulfovibrio baculatus
atctgatcgctcgtgaaaaaaaggccgaagtctctgcacgctattacctttttgaaggaggagggtatgagtcttagcagacgtgagttcgtaaaactgtgctccgcaggtgtcgccggattgggaatttcccagatttatcatccgggcatcgtgcacgccatgaccgaaggggccaaaaaagctccggtcatctgggtacagggacagggttgtactggttgctccgtttctcttctcaatgcagtccatcccagaatcaaggagattctgctggatgtgatcagccttgagttccatcccaccgtcatggcaagtgaaggtgagatggcattggcgcacatgtacgaaattgctgaaaagtttaacggcaactttttcttgctggtggaaggtgccatccccaccgccaaggaaggtcgctactgcattgtcggtgaaactctggatgccaaggggcatcatcatgaagtcaccatgatggaactgatccgggatctggcacccaagtccctggccaccgtggccgtgggcacttgttccgcttacggaggcattcccgccgccgaaggcaacgtcaccggctccaagagcgtgcgtgatttctttgccgatgaaaagatcgaaaaactgctggtcaacgtgcccggatgtccgccccatccggactggatggtcggcacgctggttgccgcatggagccacgtcctgaatcccaccgagcatcccctgcccgaactggatgacgacggacgcccgctgctgttctttggcgacaacatccacgagaactgtccgtatcttgataaatacgacaactccgaattcgcggaaaccttcaccaagcccggttgcaaggccgaacttggttgcaagggtccgtccacctatgccgattgcgccaagcgtcgctggaacaacggcatcaactggtgtgtcgagaacgccgtgtgtatcggctgtgtggaaccggactttccggacggaaagtctcctttctatgtagcggaataatcaaggaggacgcacgtgtcacaagcagctactcccgcagctgacgggaaagttaagatttccatcgatccgttgactcgggttgaaggtcatctcaagattgaggttgaagtcaaggacggcaaggtcgtcgatgccaagtgttccggcggaatgttccgcgggttcgagcagattctgcgcggccgcgatcccagggattcttcccagatcgtacagcgtatctgcggcgtgtgccccacggcgcactgtacggcttccgtcatggcccaggacgacgcctttggcgtcaaagtaaccaccaacggccgcatcacccgtaacctgatcttcggcgccaactatctgcagtctcatattctgcatttctatcacctggccgccctggattacgtcaagggtcccgatgtatctccctttgttccccgttacgccaatgcggatctcttgacggatcgcatcaaggacggagccaaggccgatgcaaccaacacctacggcttgaaccagtacctgaaggcgcttgaaatccgccgcatctgtcatgagatggtcgccatgtttggcggtcgcatgcctcatgttcagggcatggtcgtgggcggtgcaaccgagattcccacggcggacaaagtcgcggaatacgcggcccgcttcaaggaagtccagaagttcgtgatcgaggaatatctgcctctgatctacaccctaggttccgtttacacggatctgttcgagaccggcatcggctggaagaacgtcatcgccttcggcgttttccccgaagacgatgattacaagaccttcctgctcaagcccggcgtatatatcgacggaaaggacgaggaattcgattccaagctggtcaaggaatatgtcggacattccttctttgaccattccgctcccggcggtctgcactacagcgtcggtgaaacgaatcccaacccggacaaacccggtgcgtacagcttcgtcaaggctccccgttacaaggacaagccctgcgaagtcggtccgctggcccgcatgtgggtccagaaccccgagctcagccccgttggccagaaactgctcaaggaactttacggcatcgaagccaagaagttccgcgatctgggcgacaaggctttctccatcatgggccgccacgtgctcgtggctgaagaaacctggcttaccgcagtggccgttgaaaaatggctcaagcaggttcagcccggcgccgagacctacgtcaagtccgagattccggacgccgccgaaggcaccggattcactgaagcaccccgtggcgctttgctgcattacctcaagatcaaagacaagaagatcgagaactatcagatcgtgtctgcgactctctggaatgccaaccccagggatgacatgggacagcgcggcccgatcgaggaagccctcatcggtgtgccggttcccgacatcaagaatcccgttaatgtggggcgcctggtgcgctcctacgacccgtgactgggctgtgccgtgcacgtgctgcacgctgagaccggtgaagaacacgttgtcaacattgactaacgcggattgactgaa

SEQ ID NO: 7 - DNA, 1544 bp, Trichomonas vaginalis
atcgtcgtattgaaatttttactgattatccaagcaaattttagtaataaatgcgtatttgcaatatgataaaaatattggatttcgtttttaaaatttgtgaaactagaaattttccctagttcatttttcatcaaatgttagcgagttcagcaacagcaatgaaaggatttgcaaatagtctcagaatgaaagactatagctcaacaggtatcaattttgatatgacaaaatgcataaattgccaatcatgtgtcagagcatgcacaaacatcgcaggtcaaaatgttcttaagtctttaacagtcaatggtaaatcagtggtacagacagttacaggaaaaccacttgcagaaactaattgcatctcatgtggacagtgtacgttaggttgtccaaagtttactatctttgaagctgacgcaattaatccagtgaaggaagttcttacaaagaaaaacggaagaattgcagtttgccaaattgcacctgcaatcagaattaatatggcagaagccctaggtgttccagctggaacaatctcattaggtaaggttgttactgcattaaagcgtcttggctttgattacgtctttgatacaaactttgcagcagatatgacaattgttgaagaagcaacagaattagttcagcgtctctcagacaaaaacgccgttcttccaatgtttacatcatgctgtcctgcttgggtaaactatgttgagaaatcagatccatctctgattccgtacttaagctcatgcagatctccaatgagtatgctttcttctgtaatcaaaaatgtgttcccaaagaagattggaacaacagctgataagatttataacgttgctattatgccatgcacacgaaagaaggatgaaatccaaagatcacaattcacaatgaaagatggaaagcaagagacaggtgcagtcttaacatccagagaattagccaagatgatcaaagaagcaaaaatcaacttcaaagagttaccagatacaccatgcgataacttctactcagaggcttctggtggtggtgcaatcttttgtgcaactggtggtgttatggaagctgcagttcgttctgcttacaaattcttgacaaagaaggaactcgcaccaattgaccttcaagatgttcgaggtgttgcaagtggtgtcaaacttgctgaagttgatattgcaggtacaaaagtcaaagttgcagttgctcacggcataaagaatgcaatgactttgatcaaaaagatcaaatctggtgaggaacaatttaaagatgttaagtttgttgaagtcatggcttgtccaggcggctgtgttgttggcggtggttctccaaaggccaagacaaagaaagctgttcaagccagactcaacgctacatattctattgataaatcatcaaaacatagaacatctcaagacaatccacagcttttgcaattgtacaaggaatcgttcgagggtaagtttggtggccacgtagctcaccaccttttacacactcattacaagaacagaaaagttaatccatag

SEQ ID NO: 8 - DNA, 20 bp, Artificial Sequence
  A synthetic primer
actgtcaccattggcgatgc

SEQ ID NO: 9 - DNA, 20 bp, Artificial Sequence
  A synthetic primer
tgccttgctggacgacattc

SEQ ID NO: 10 - DNA, 20 bp, Artificial Sequence
  A synthetic primer
agtatatgaccgaccgcata

SEQ ID NO: 11 - DNA, 21 bp, Artificial Sequence
  A synthetic primer
ttcgtaaggatacccggttgc

(19) United States
(12) Patent Application Publication
(10) Pub. No.: US 2007/0009437 A1
(43) Pub. Date: Jan. 11, 2007

(54) Intracellular trapping of radionuclides by enzyme-mediated reduction

(76) Inventors: Fangyu Peng, Bloomfield Hills, MI (US); King Li, Bethesda, MD (US); Sunil D. Pandit, Gaithersburg, MD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/390,965

(22) Filed: Mar. 28, 2006

Related U.S. Application Data
(63) Continuation of application No. PCT/US04/31843, filed on Sep. 29, 2004.
(60) Provisional application No. 60/507,607, filed on Sep. 30, 2003.

(51) Int. Cl. A61K 51/00; C12Q 1/68

(52) U.S. Cl. 424/1.69; 435/6

(57) ABSTRACT

Enzyme-mediated intracellular trapping of a radionuclide in a target cell is achieved by transfecting the target cell with a transgenic vector encoding a microbial hydrogenase expressible in the target cell and exposing the transfected target cell with a radionuclide. The transgenically expressed microbial hydrogenase catalyzes the reduction of the radionuclide. The reduced radionuclide becomes trapped intracellularly where its emissions can be detected in radioscintigraphy applications. In addition, emissions from an intracellularly trapped radionuclide can be cytotoxic to the cell and therefore useful in radiotherapy applications. As a reporter, a microbial hydrogenase encoding nucleic acid can be included in a vector along with a transgene, both under the control of the same promoter. The detection of emissions from intracellularly reduced and trapped radionuclide can be used to monitor transgene expression.

This application is a rule 111 (a) continuation of PCT application Ser. No. PCT/US2004/031843, filed Sep. 29, 2004, which claims priority to Provisional Application Ser. No. 60/507607, filed Sep. 30, 2003, the contents of which are incorporated herein in their entirties.

### GOVERNMENT FUNDING

The invention described herein was developed with the support of the Department of Health and Human Services. The United States Government has certain rights in the invention.

### FIELD OF THE INVENTION

This invention in the field of molecular biology relates to the intracellular trapping of radionuclides useful in radioscintigraphy and radiotherapy.

### BACKGROUND OF THE INVENTION

Radionuclides used in nuclear medicine are useful for treating and imaging diseased tissue, especially cancer. Although the major medical utilization of radionuclides has been for imaging and diagnostics, there is an increasing interest in radionuclides as cancer therapeutics.

Clinical applications of therapeutic radionuclides have progressed in relatively new and developing areas, such as radioimmunotherapy, peptide therapy, intravascular therapy to prevent restenosis, radiation synovectomy, and bone malignancy therapy. The hope that targeted delivery of radionuclides to tumor sites, e.g., antibody conjuged radionuclides or other molecules that can specifically attach to tumor cells, allows effective and well-tolerated systemic therapy. Radiolabeled molecules that bind to specific cell surface components provide one successful approach to tumor imaging and therapy. Examples are OctreoScan® for imaging and potentially treating neuroendocrine neoplasms, CEAScan® and OncoScint® for imaging colorectal and ovarian cancers, and Bexxar® and Zevalin® for treating certain lymphomas.

Other examples include U.S. Pat. No. 5,730,982 to Scheinberg, which describes monoclonal antibodies containing the hypervariable region of M195 conjugated to a cytotoxic radionuclide specific for cancerous bone marrow. Similarly, U.S. Pat. No. 5,320,956 to Willingham et al., describes radionuclide conjugated monoclonal antibodies (secreted by a hybridoma with ATCC No.: HB 10570) used in the diagnosis of ovarian, esophageal and cervical carcinomas.

The present invention provides a novel method of localizing radionuclides through enzyme mediated intracellular trapping for radioscintigraphy or radiotherapy without the need for conjugation to a delivery agent such as a peptide or antibody.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The invention provides a method for intracellular radionuclide trapping wherein a mammalian target cell is transfected with a transgenic vector having a nucleic acid encoding a microbial hydrogenase (reductase) and operatively linked promotor. The transfected cell is treated with a radionuclide which diffuses into the transfected target cell. The transgenically expressed microbial hydrogenase mediates the reduction of the radionuclide. The reduced radionuclide becomes intracellularly trapped. Radionuclide trapping is useful for radioscintigraphy (positron emission topography or single photon emission computerized tomography) or radiotherapy (emission of radioation cytotoxic to the target cell) of mammalian cells, especially human cancer cells.

In one embodiment of the invention, the microbial hydrogenase is Escherichia coli—Hydrogenase 3.

In another embodiment of the invention, the microbial hydrogenase is Desulfovibrio desufuricans—Hydrogenase.

In another embodiment of the invention, the microbial hydrogenase is Trichomonas vaginalis—Hydrogenase.

The preferred radionuclide used for the invention is Technitium-99m, however, Technitium-94m, Rhenium 186, and Rhenium 188 can also be used. Other positron-emitting, β-emitting, γ-emitting radionuclides alone or in combination are useful.

Another aspect of the invention is a method for reporting transgene expression is also provided wherein a target cell is transfected with a vector encoding a transgene and a microbial hydrogenase each under the control of a first and second promoter, respectively, that are recognized by an RNA polymerase. The transfected target cell is contacted with a radionuclide that diffuses into the transfected cell. The radionuclide is reduced and intracellularly trapped in a reaction mediated by the expressed microbial hydrogenase. Transgene expression can be reported by detecting emissions from the trapped radionuclide.

Also provided is a mammalian cell complex that includes a mammalian cell transfected with a vector encoding a microbial hydrogenase and an operatively linked promoter, and a radionuclide that diffuses into the transfected mammalian cell.

### BRIEF DESCRIPTION OF THE DRAWINGS

FIG. 1 is a drawing that shows (A) the gene structure of the Escherichia coli hydrogenase 3 operon (hyc-E encoding the large subunit of the hydrogenase and hyc-G that encodes the small subunit of the dydrogenase); (B) the Desulfovibrio desulfican hydrogenase operon, and (C) the Trichomonas vaginalis hydrogenase operon.

FIG. 2 is a map of a plasmid constructed with a nucleic acid segment that encodes a microbial hydrogenase. This plasmid had the following segments: CMV promoter (bases 7-594), CMV forward priming site (bases 544-564), T7 promoter/priming site (bases 638-657), CMV multiple cloning site (bases 664-713), myc epitope (bases 719-748), 6× His tag (bases 764-782), SV40 polyadenylation sequence (bases 803-933), Zeocin™ resistance gene (bases 1063-1437, complementary strand), EM7 promoter (bases 1456-1510, complementary strand), SV40 early promoter (bases 1457-1869, complementary strand), EF-1α promoter (bases 1885-3051), EF-1α forward priming site (bases 2999-3019), EF-1α multiple cloning site (bases 3062-3126), V5 epitope (bases 3127-3168), 6× His tag (bases 3178-3195), BGH reverse priming site (bases 3218-3235, complementary strand), BGH polyadenylation sequence (bases 3224-3447), and pUC origin (bases 3521-4194).

FIG. 3 illustrates an agarose gel electrophoresis of the restriction digestion of a plasmid that was constructed with a sequence that encodes a microbial hydrogenase.

FIG. 4 provides a schematic diagram of the bacterial induction vector pETDUET1/HydA/HydB.

FIG. 5 provides a schematic diagram of the bacterial induction vector pACYCDuet/DsbA-HydAF that provides periplasmic targeting.

FIG. 6 provides a schematic diagram of the bacterial induction vector pETDuet1/DsbC-HydB that provides periplasmic targeting.

FIG. 7 provides a schematic diagram of the mammalian induction vector pBudCE4.1/HydA/HydB.

FIG. 8 provides a schematic diagram of the mammalian induction vector pBudCE4.1/Mito-HydB-myc/Mito-HydA-myc.

FIG. 9 provides a schematic diagram of the mammalian induction vector pBudCE4.1/Mito-HydA/Mito-HydB.

FIG. 10 provides results of a technetium reduction assay involving cells transfected with various vectors of the invention at the time points indicated along the x-axis. The y-axis provides counts per minute (cpm). This bar graph provides cpm observed at each time point for pET-Duet+pACYCDuet (first bar on the left at each time point), pETDuet/HydA/HydB (second bar), pACYC-DsbA-HydA+pETDuet/DsbC-HydB (third bar), pACYC-DsbA-HydA alone (fourth bar) and pETDuet/DsbC-HydB alone (fifth bar). This data is representative of several experiments.

FIG. 11 provides technetium reduction assay results for cells transfected with various vectors of the invention at the time points indicated along the x-axis. The y-axis provides counts per minute (cpm) for one million cells transfected with pET-Duet+pACYCDuet (first bar on the left at each time point), pETDuet/HydA/HydB (second bar), pACYC-DsbA-HydA+pETDuet/DsbC-HydB (third bar), pACYC-DsbA-HydA alone (fourth bar) and pETDuet/DsbC-HydB alone (fifth bar). This data is representative of several experiments.

FIG. 12 graphically illustrates 99mTc trapping observed in aerobically grown cultures of BL21(DE3) following induction with 100 mM IPTG. These results are reported as normalized counts per minute at the time points following induction that are shown along the x-axis. The normalized counts per minute were calculated by dividing the experimentally observed CPM by the number of CPM expected for the entire 10 mls of media minus the CPM for control bacteria divided by CPM expected in 10 mls of media (eCPM/ncCPM)-(cCPM/ncCPM). Results shown are for pETDuet/HydA/HydB (first bar on the left at each time point), pACYC-DsbA-HydA alone (second bar), pETDuet/DsbC-HydB alone (third bar) and pACYC-DsbA-HydA+pETDuet/DsbC-HydB (fourth bar).

FIG. 13 provides a reverse transcriptase-polymerase chain reaction (RT-PCR) analysis of HydA (left) and HydB (right) expression from a representative induction of BL21 (DE3) Star (Invitrogen) E coli. Cells were grown to an optical density at 260 nm (OD₂₆₀) of 0.6 and induced for 0, 1, 2 and 3 hrs with 1 mM IPTG. The anticipated band was 300 bp. No Reverse Transcriptase (No-RT) controls, and a 250 bp marker ladder (M) are also shown. RT-PCR results confirmed that expression of both HydA and HydB mRNA was induced.

FIG. 14 illustrates the proteins expressed in cells transfected with different vectors of the invention. Total cell protein was extracted from induced BL21 (DE3) Star cells with the pEt Duet/HydA/HydB vector. Proteins were electrophoretically separated run on a 10% SDS/Glycine gel and stained with Commassie blue. This protein expression analysis indicated that the HydA and HydB gene products were likely over-expressed.

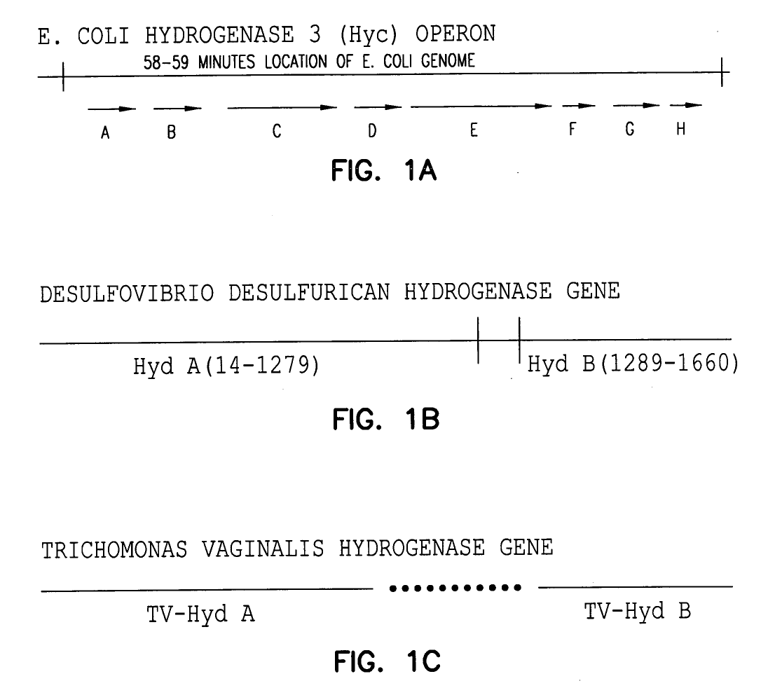

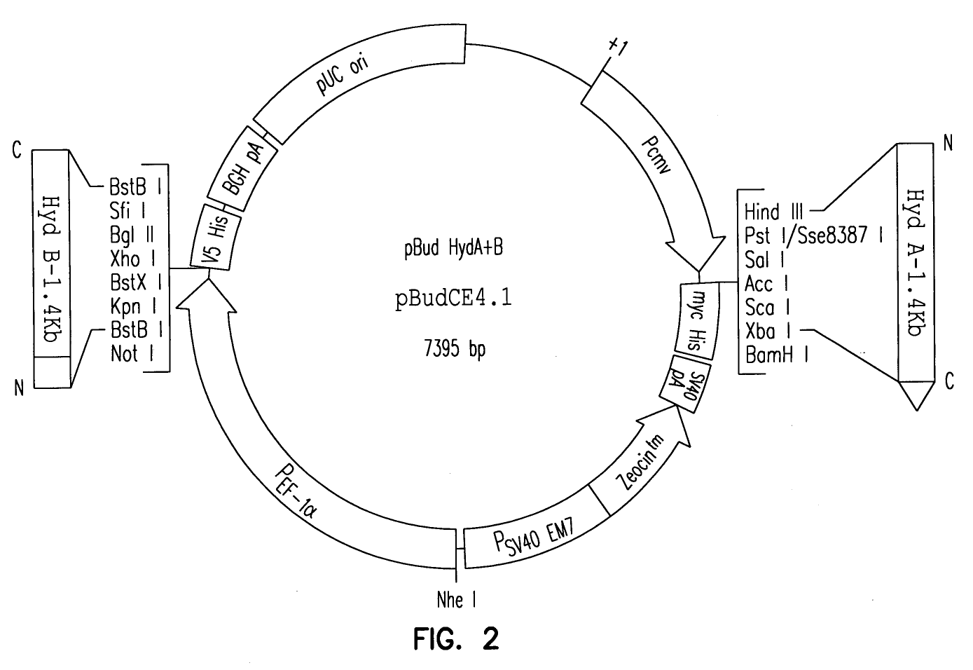

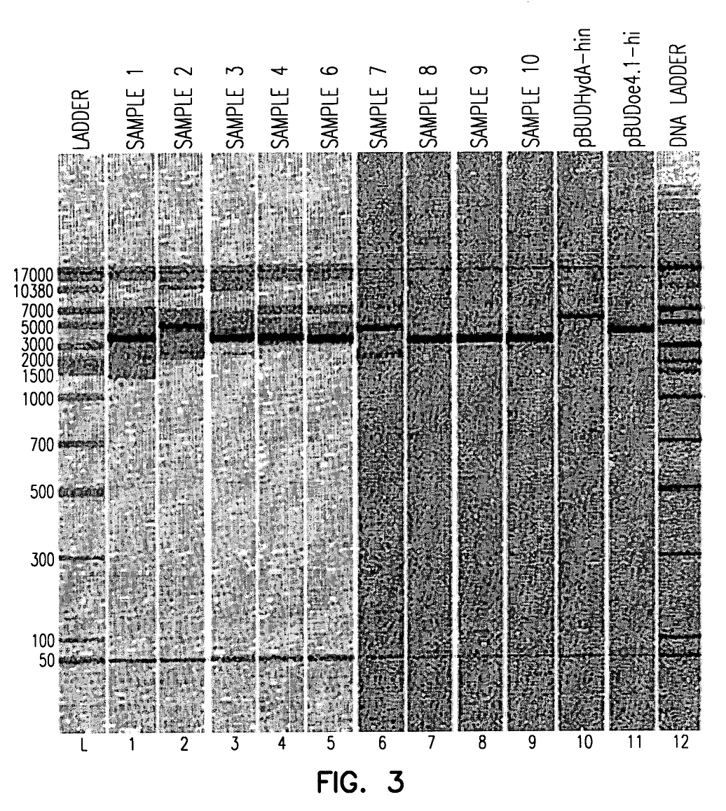

The foregoing figures are provided for illustration purposes and in no way limit the scope of the claims.

### DETAILED DESCRIPTION OF THE INVENTION

Intracellular trapping of radionuclides within a target cell (e.g., a human cancer cell) is achieved by the intracellular reduction of the radionuclide by a reaction that is mediated by a microbial hydrogenase (reductase). In an exemplary embodiment, a target cell is transfected with a transgenic vector including a nucleic acid segment encoding a microbial hydrogenase (reductase) expressible in the target cell and one that is catalytically active. The transgenic vector preferably also includes a promoter operatively linked to the nucleic acid segment encoding the microbial hydrogenase (reductase) and is a promoter suitable to control expression of the nucleic acid segment. The transfected target cell is contacted with a radionuclide which diffuses into the target cell. The transgenically expressed and catalytically active microbial hydrogenase mediates the reduction of the radionuclide. The reduced radionuclide is intracellularly trapped where it reacts non-specifically with intracellular proteins and nucleic acids (Breuer M (1999) Angiology 7:563-71). The trapped radionuclide can a) be cytotoxic to the target cell by disrupting cellular function through non-specific inhibitory binding and radiation emissions and is therefore useful as a radiotherapeutic or b) become trapped and linger within the target cell while emitting radiation detectable by radioscintigraphy (e.g., positron emission tomography or Single Photon Emission Computerized Tomography) (Pantuck A J (2002) J Urol. 168(3):1193-8).

Radionuclides Useful in the Invention

Several radionuclides and radiopharmaceuticals are available for radiotherapy and radioscintigraphy of neoplasms. They range from classical agents such as sodium iodide (Na-¹³¹I), thallous chloride ([²⁰¹TlCl], and gallium citrate (⁶⁷Ga-citrate) to highly selective positron-emitting reporter gene detection systems (Vallabhajosula S (2001) In Nuclear Oncology. I Khalkhali et al., Eds. Lippincott Williams & Wilkins, Philadelphia, Pa. pp. 31-62; Iyer M et al. (2001) J Nucl Med 42, 96-105).

Selection of a radionuclide is based on properties such as emission type, linear energy transfer, physical half-life; dictated by the character of the disease tissue (e.g., solid tumor or metastatic disease), and by the carrier used to selectively transport the radionuclide to the target tissue (if applicable).

Examples of radionuclides useful to implement the invention are shown in Table 1 below:

TABLE 1
Exemplary Radionuclides Useful For Radiotherapy And/Or Radioscintigraphy

| | ATOMIC | MASS | HALF- | | | |
|---|---|---|---|---|---|---|
| ELEMENT | NUMBER | NUMBER | LIFE | | EMISSION TYPE | REFERENCE |
| Chromium | 24 | 51 | 27.8 | days | Gamma | Lanciano R et al., (1998) Semin Oncol 25(3): |
| | | | | | | 361-371 |
| Gallium | 31 | 67 | 78 | hours | Gamma | Carney PL et al., (1989) J Nucl Med 30: 374-384 |
| Iodine | 53 | 123 | 13 | hours | Gamma | Robbins J et al., (2000) Rev Endocr Metab Disord |
| | | | | | | 1(3): 197-203 |
| Iodine | 53 | 125 | 59.4 | days | Gamma | Robbins J et al., (2000) Rev Endocr Metab Disord |
| | | | | | | 1(3): 197-203 |
| Iodine | 53 | 131 | 8 | days | Beta | Robbins J et al., (2000) Rev Endocr Metab Disord |
| | | | | | | 1(3): 197-203; |
| | | | | | | Sgouros G, et al., (2003) J Nucl Med 44(2): 260-8 |
| Indium | 49 | 111 | 2.8 | days | Gamma | Eckelman WC et al., (1980) Cancer Res 40: 3036-3042 |
| Selenium | 34 | 75 | 120 | days | Gamma | Eckelman WC et al., (1980) Cancer Res 40: 3036-3042 |
| Technetium | 43 | 99m | 6 | hours | Gamma | Leung JW et al., (2002) Radiol Clin North Am 40(3): |
| | | (metastable | | | | 467-82 |
| | | Tc-99) | | | | |
| Technetium | 43 | 96 | 4.28 | days | Gamma | Di Rocco RJ et al., (1992) J Nucl Med 33(6): 1152-9 |
| Technetium | 43 | 94 | 293 | minute | Gamma | Hiromatsu Y et al., (2000) Intern Med 39(2): 101-6 |
| Technetium | 43 | 94m | 52.5 | minutes | Beta | Smith MF et al., (2001) Med Phys 28(1): 36-45 |
| | | (metastable | | | | |
| | | Tc-94) | | | | |
| Thallium | 81 | 201 | 73 | hours | Gamma and Beta | Connolly LP et al., (2002) Clin Nucl Med 27(2): 117-25 |
| Rhenium | 75 | 186 | 3.7 | days | Beta | Podoloff DA et al., (2002) Curr Pharm Des 8(20): |
| | | | | | | 1809-14 |
| Rhenium | 75 | 188 | 17 | hours | Beta | Knapp FF Jr. et al., (1998) Cancer Biother Radiopharm |
| | | | | | | 13(5): 337-49 |
| Samarium | 62 | 153 | 46.3 | hours | Gamma and Beta | Han SH, et al. (2002) J Nucl Med 43: 1150-1156 |
| Iridium | 77 | 192 | 73.8 | days | Gamma and Beta | Mazeron JJ et al., Radiother Oncol (1991) 21(1): 39-47 |
| Strontium | 38 | 89 | 50.5 | days | Beta | Joensuu H et al., (1999) Acta Oncol 38 Suppl. 13: 75-83 |
| Lead | 82 | 212 | 10.6 | hours | Beta | Kumar K (1989) A J Chem Soc Chem Commun 3: |
| | | | | | | 145 146 |
| Bismuth | 83 | 212 | 60.5 | minute | Beta and Alpha | Kumar K (1989) A J Chem Soc Chem Commun 3: |
| | | | | | | 145 146 |
| Yttrium | 39 | 90 | 64 | hours | Beta | Hohenstein M et al., (2002) Semin Oncol Nurs 18 Suppl. |
| | | | | | | 1: 10-5 |

Two major technologies used to detect emissions from radionuclides are preferred in radioscintigraphy embodiments of the invention: Single Photon Emission Computerized Tomography (SPECT) carried out with a gamma camera, employing radionuclides in which a single high energy photon is directly emitted (Budinger T F et al., (1992) J Neural Transm 36(suppl): 3-12); and Positron Emission Tomography (PET), in which a positron is emitted from the radionuclide (Phelps M E et al., (2002) Proc Natl Acad Sci USA 97:9226-9233). For positron imaging, positrons are emitted from nuclei of proton-rich isotopes, and eventually interact with electrons. Annihilation occurs, and the mass of the electron and positron is converted into two γ-rays that travel outward from the site of annihilation at about 180° to one another. Scintillation crystals composed of different materials are used to capture the γ-rays for both SPECT and PET. The collection of many events allows reconstruction of the source of the emissions. PET data can be corrected for attenuation, which occurs because some gamma rays do not traverse through all of the surrounding tissue. SPECT data is more difficult to correct for attenuation effects. PET is about 10-fold more sensitive than SPECT, primarily because the SPECT cameras use collimators that reject many of the counts from the source (Fleming J S et al., (1996) J Nucl Med 37:1832-1836).

Microbial Hydrogenases (Reductases)

Lovley et al. in 1993 and Macaskie et al. in 1991 suggested the use of microbial hydrogenase mediated reduction of radionuclides as a biotechnological method of treating radionuclide contaminated effluents (Lovley D R, et al. (1993) Ann Rev Microbiol 47:263-290; Macaskie L E, et al., (1991) CRC Crit Rev Biotechnol 11:41-112). This was later demonstrated in experiments showing the ability of resting cells of the Fe(III)-reducing bacteria Shewanella putrefaciens and Geobacter metallireducens to reduce technetium, Tc(VII), using lactate and acetate as reducing agents (Lloyd J R et al., (1996) Appl Environ Microbiol 62:578-582). Later studies showed that Tc(VII) reduction is not exclusive to Fe(III)-reducing bacteria as demonstrated by the ability of E. coli (Lloyd J R, et al., (1997) J Bacteriol 179:2014-2021) and the sulfate reducing bacterium Desulfovibrio desulfuricans (Lloyd J R et al., (1998) Geomicrobiol J 15: 43-56) to reduce and precipitate Tc(VII) out of effluent.

The invention utilizes the catalytic properties of microbial hydrogenases to intracellularly mediate the reduction of radionuclides. A vector encoding a microbial hydrogenase is used to transfect a target cell. A nucleic acid segment encoding the microbial hydrogenase is preferably one that is expressible in non-native and preferably mammalian, cells. The microbial hydrogenase is preferably one that retains its native catalytic properties after being expressed in the target cell. For example, a microbial hydrogenase of E. coli suitable for catalyzing Tc(VII) reduction is hydrogenase 3, a component of the formate hydrogenlyase complex (FHL), encoded by the hyc operon (Rossman et al., (1991) Mol Microbiol 5:280-2814). E. coli hydrogenase 3 is encoded by the hyc operon located at 58-59 minutes on the E. coli chromosome. Native hyc includes subunits A through I: subunit A is a negative regulator, subunit B is the small subunit of the formate dehydrogenase, subunits C, D, and F are membrane proteins that provide structural support for the protein but have no known catalytic activities, subunit E is the large subunit of hydrogenase 3 Membrane protein, and G is the small subunit of hydrogenase 3; subunit I is a protease responsible for cleaving the several subunits into active enzyme. The hycD, hycE, and hycG genes of the hyc operon are particularly important in forming active hydrogenase 3. Mutation studies showed that mutations in any one of these genes impaired Tc(VII) reduction as demonstrated by a drop in Tc(VII) precipitation by 20% versus the precipitation observed after reduction by wild-type (Lloyd J R et al., (1999) Biotechnol & Bioengin 66(2): 122-130).

Hydrogenases found in the sulfate reducing bacteria of the Desulfovibrio genus (e.g., D. desulfuricans, D. vulgaris, D. fructosovorans, D. gigas, D. baculatus) as shown in Table 2 below, are also useful for intracellular radionuclide reduction and the nucleic acids encoding them can be included in a transgenic 30 vector used for transfecting a target cell. Hydrogenases found in the Trichomonas genus (e.g. T. vaginalis) can also be used for intracellular reduction of radionuclides.

TABLE 2
Exemplary Microbial Hydrogenases (Reductases) Useful For Catalyzing Radionuclide Reduction

| Hydrogenase (reductase) | Source | SEQ ID No. | Reference |
|---|---|---|---|
| Hydorgenase 3 (Hyc) | Escherichia coli | SEQ ID No. 1 | Lutz, S. et al., Mol. Microbiol. 4(1): 13-20. |
| Periplasmic [Fe]-hydrogenase | Desulfovibrio vulgaris | SEQ ID No. 2 | Voordouw, G et al., (1989) J. Bacteriol. 171(7): |
| | | | 3881-3889. |
| Periplasmic [NiFe]-hydrogenase | Desulfovibrio desulfuricans | SEQ ID No. 3 | Ringbauer, JA Jr., et al., [NiFe] hydrogenase of |
| | | | Desulfovibrio desulfuricans. Submitted (DEC |
| | | | 2000) to the EMBL/GenBank/DDBJ databases; |
| | | | Lloyd, JR et al., (1999) Biotechnol & Bioengin |
| | | | 66(2): 122-130 and (1999) Appl Environ |
| | | | Microbiol. 65(6): 2691-6. |
| Periplasmic [NiFe]-hydrogenase | Desulfovibrio fructosovorans | SEQ ID No. 4 | De Luca G et al., (2001) Appl Environ Microbiol |
| (Hyd) | | | 67(10): 4583-7; Rousset, M, et al., (1990) Gene |
| | | | 94: 95-101. |
| Periplasmic [NiFe]-hydrogenase | Desulfovibrio gigas | SEQ ID No. 5 | Li C, et al., (1987) DNA 6(6): 539-551. |
| Periplasmic [NiFeSe]- | Desulfovibrio baculatus | SEQ ID No. 6 | Menon, NK, et al. (1987) J. Bacteriol. 169(12): |
| hydrogenase | | | 5401-4429. |
| Hydrogenosomic [Fe]- | Trichomonas vaginalis | SEQ ID No. 7 | Bui, ET and Johnson, PJ, (1996) Mol & Biochem |
| hydrogenase (TvHyd) | | | Parasitol 76: 305-310. |

Nucleic acid segments that encode hydrogenases suitable for reducing radionuclides can also be cloned from iron reducing bacteria such as Geobacter sulfurreducens, Geobacter metallireducens, Acidophillic bacteria (genus Thiobacillus), Rhodobacteter capsulates, Methanobacterium thermoautotrophicum, Shewanella putrefaciens, Shewanella oneidensis for inclusion in a transgenic vector used to transfect a target cell.

The microbial hydrogenase selected should also be oxygen stable such as the hydrogenases of D. vulgaris, D. desulfuricans listed in Table 2 above. Also, suitable microbial hydrogenases will remain catalytically active at temperatures between about 20° C. to about 37° C.

Transgenic Vectors Including Microbial Hydrogenase (Reductase)

Exemplary vectors include a nucleic acid segment that encodes a microbial hydrogenase suitable for catalyzing the reduction of radionuclides like hydrogenase 3 from E. coli (MC4100 strain) (Lloyd J R, et al., (1999) supra). A mammalian expression vector including a nucleic acid segment encoding a functional E. coli hydrogenase 3 can be constructed by amplifying hydrogenase 3 subunits E and G of the enzyme using Elongase PCR and cloning the amplicons into pCR2.1-TOPO (Invitrogen). The amplified fragments can then be subcloned into mammalian expression vector pcINeo (Promega Corporation, Madison, Wis.). The pCI-neo mammalian expression vector carries the human cytomegalovirus (CMV) immediate-early enhancer/promoter region useful in promoting constitutive expression of cloned DNA inserts in mammalian target cells. The pCI-neo vector also contains the neomycin phosphotransferase gene, a selectable marker for mammalian cells. The pCI-neo vector can be used for transient expression or for stable expression by selecting transfected target cells with the antibiotic G-418 (available from A.G. Scientific or from BD Biosciences Clonetech).

Other exemplary vectors can encode a periplasmic [NiFe]-hydrogenase from D. desulfuricans, or the [Fe]-hydrogenase from D. vulgaris (e.g., GenBank accession number AF331719), both of which are suitable for catalyzing the intracellular reduction of radionuclides.

The hydr subunit (hydA) and the hydrogenase small subunit precursor (hybB) can be Elongase PCR cloned into pCR2.1-TOPO (Invitrogen). pTopo-DdhydA can be digested with Hind III and XbaI and subcloned into mammalian dual expression vector pBud-CE4.1 (Invitrogen) (pBud-DdHydA). pTopo-DdhybB can be digested with Not I and KpnI and also subcloned into pBUD CE4.1 (pBud-DdHybB).

Other useful transgenic constructs include either or both subunits of the hydrogenase encoding gene of the eukaryotic parasite Trichomonas vaginalis. Subunits A and B (TvhydA and TvhydB) of T. vaginalis were each Elongase PCR cloned into pCR2.1-TOPO (Invitrogen) using primers and templates obtained from the University of California at Los Angeles. pTopo-TVhydA was digested with Hind III and XbaI and subcloned into mammalian dual expression vector pBud-CE4.1 (Invitrogen) (pBud-TvHydA). pTopo-TVhyd B was digested with Not I and KpnI and also subcloned into pBUD CE4.1 (pBud-TvHydA).

To generate an expression vector including both expressible subunits the TV-hydB gene was amplified with PCR primers containing 5′ BstB I or Sfi I restriction sites. The amplified DNA products were then digested with BstBI or Sfi I and cloned into pBud-hydA at the BstBI or SfiI sites under an EF1a promoter to obtain pBud-Tvhyd A&B. pBud-Tvhyd A&B has TvhydA under the control of the CMV promoter and TvhydB PCR DNA fragment or cDNA under the control of EF-1a promoter.

Delivery of Transgenic Vector into Target Cell

Electroporation

Electroporation, also known as electropermeabilization, is popularly used for transfection of cell suspensions. Forcep electrodes are used for in vivo electroporation aimed at various organs and tissue types (Muramatsu et al. (1998) J. Mol. Med. 1: 55-62). Electroporation entails exposing cells to short intense electric field pulses thereby inducing a transmembrane potential. The applied field induces temporary structural changes in the cell membrane, creating pathways from the extracellular space into the cell interior (Jaroszeski et al. (2000) Meth. Mol. Med. 37:173-186).

Currently, transmembrane induction voltages between 0.5 and 1 V minimum potential are used for most mammalian cell types. Induction potentials above this threshold can cause irreversible membrane damage and cell death (Teissié et al. (1992), In: Charge and Field Effects in Biosystems (Allen, M. J et al. eds.) Birkhauser, Boston, 3: 285-301); Teissie et al. (1982) Science 216:537-538).

Apart from the extent of membrane permeabilization, determined primarily through pulse duration and voltage, other factors control the intake of exogenous DNA by the cell. Most electroporation-mediated exogenous DNA transport is through electrophoresis rather than solely by membrane permeability (Klenchin et al. (1991) Biophys. J. 60:804-811, Wolf et al., Biophys. J. 66:524-531). Adding DNA immediately after the pulse usually results in lower transfection efficiency than when the DNA is added prior to the pulse. Shielding the charge of DNA by cations also reduces transfection efficiency (Anderson et al. (1989) 180: 269-275).

Most of the DNA entering the cell does so during the pulses by way of the electric field created across the membrane. As such, the transfection efficiency is proportional to the integral of the extent of membrane permeabilization with respect to the pulse time. For simple rectangular pulses or exponentially decaying pulses, the time integral is the pulse length T, which is usually much greater than the membrane relaxation time. If the permeabilized area of the cell is limited to polar regions, as is normally the case, the extent of membrane permeabilization (in terms of the number, density, and size of electropores) is approximately proportional to E-E_{b}, where E_{b} is the pulse field strength needed to produce the membrane breakdown voltage V_{b} (Hibino et al. (1991) Biophys. J. 59: 209-220). Thus the transfection efficiency is roughly proportional to (E-E_{b})T (Hui et al. (2000) Meth. Mol. Med. 37: 157-171).

Nucleic Acid-Coated Microprojectile Bombardment

This method involves propulsion of nucleic acid-coated microprojectiles (preferably DNA) into target cells (Sanford et al. (1988) Particle Sci. Technol. 5: 27-37). Gold particles are particularly preferred.

A commercially available device (Biolistic PDS-1000; from Du Pont) uses a gunpowder discharge to impart momentum to coated projectiles. When performed in vitro, target cells are placed in a vacuum chamber during bombardment to minimize air impedance of particle flight.

Williams et al., (2000) Proc. Natl. Acad. Sci. USA 88:2726-2730, disclose a device for microprojectile bombardment to introduce and express exogenous nucleic acids directly in intact tissue of the living mouse. This device, which can be configured to be hand-held, uses a helium discharge system and a disc macrocarrier for microprojectiles. The use of helium gas permits precise regulation of particle velocity and configured such that the helium discharge impelling the microprojectiles is deflected away from the tissue, minimizing damage from the resulting shock wave. The helium discharge drives the macrocarrier through a 0.8 cm flight path to a stopping screen that arrests the macrocarrier disc but is permeable to nucleic acid coated microprojectiles which are permitted to strike the target tissue. Tissue becomes bombarded with coated gold particles (available from Alfa, Ward Hill, Mass.) having a range diameter between 1 and 3 μm or between 2 to 5 μm when tungsten particles (Sylvania) are used.

Microparticles are coated with nucleic acid by sequentially mixing gold or tungsten in an aqueous slurry comprising nucleic acid (about 1 mg/mL), CaCl₂ (2.5 M) and free-base spermidine (1 M). After 10 or so minutes of incubation, the microprojectiles are pelleted and the supernatant removed. The pellet is washed once with 70% ethanol, centrifuged, and resuspended in anhydrous ethanol. The nucleic acid coated microprojectiles are spread over the macrocarrier discs and allowed to dry in a dessicator before firing. The exact specifications of the device and its use are detailed in Williams, supra, which is hereby incorporated by reference.

A person having ordinary skill in the art will recognize that other approaches may be employed to introduce the transgenic construct into target cells, such as by viral infection or lipofection.

Lipid Mediated Transfection (Lipofection)

Some of the first work on liposome delivery of endogenous materials to cells occurred some twenty years ago. Foreign nucleic acids were introduced into cells using positively charged lipids. (Martin et al., (1976) J. Cell Biol. 70: 515-526, Magee et al., (1976) Biochim. Biophys. Acta 451: 610-618, and Straub et al., (1974) Infect. Immun. 10:783-792).

Of the many methods used to facilitate entry of DNA into eukaryotic cells, cationic liposomes are among the most efficacious and have found extensive use as DNA carriers in transfection experiments. (Thierry et al., Gene Regulation: Biology of Antisense RNA and DNA, p. 147 (Erickson and Izant, Eds., Raven Press, New York, 1992).

Senior et al., (1991) Biochim. Biophys. Acta 1070:173, suggested that incorporation of cationic lipids in liposomes is advantageous because it increases the amount of negatively charged molecules that can be associated with the liposome. In their study of the interaction between positively charged liposomes and blood, they concluded that harmful side effects associated with macroscopic liposome-plasma aggregation could be avoided by limiting the dosage.

U.S. Pat. Nos. 5,695,780, 5,688,958, 5,686,620, 5,661,018, 5,651,981, herein incorporated by reference in their entirety, further elaborate the types of lipids useful in lipofection vectors and methodology used in the lipofection of nucleic acids into eukaryotic cells.

Having now generally described the invention, the same will be more readily understood through reference to the following examples which are provided by way of illustration, and are not intended to be limiting of the present invention.

### EXAMPLE I

### Trapping of Tc99m Pertechnetate in Trichomonas vaginalis by T. vaginalis Hydrogenase-Mediated Reduction

T. vaginalis (ATCC No. 30001) was cultured in ATCC LYI entamoeba medium (ATCC No. PRA-2154) at 37° C. in test tubes to a titer of 2×10⁶ cells per tube. Three test tubes were included in each group. T. vaginalis cells were incubated at 37° C. for a period of time after inoculation of 2 μci of Technetium 99m pertechnetate in each test tube. At end of incubation, the cells were harvested by centrifugation at 1000 cpm, for 10 minutes. The cellular pellet was washed three times with PBS to remove extra-cellular radioactivity. Intracellular radioactivity of the cells was counted with a gamma counter.

TABLE 3
Radioactivity in T. vaginalis

| GROUP | | INCUBATION | RADIOACTIVITY |
|---|---|---|---|
| No. | RADIONUCLIDE | TIME | (dpm) |
| 1 | 2 μCi | 30 minutes | 24904 |
| 2 | 2 μCi | 45 minutes | 45127 |
| 3 | 2 μCi | 12 hours | 1915843 |

### EXAMPLE II

### Intracellular Trapping of Radionuclides Used in Reporting

The detection of a reporter gene product is a way of “indirectly” following the expression of a gene of interest coupled to the same promoter as the reporter gene. Once a reporter gene driven by a promoter of choice is introduced into the desired tissue, expression of the reporter gene can be monitored by several conventional methods like tissue biopsy, followed by immunohistochemistry. Conventional methods to detect reporter gene expression are, however, limited by their inability to determine “non-invasively” the locations and magnitude of gene expression in living subjects over time. Approaches using green fluorescent protein (Achatz-Straussberger G (2003) Int Arch Allergy Immunol. 130(4):280-7) and firefly luciferase as reporter genes (Kogai T (2003) Breast Cancer Res Treat. 78(1):119-26) allow localization of reporter gene expression in some living animals, but monitoring of the detailed location and magnitude of reporter gene expression over time is difficult.

The choice of a promoter for reporter gene expression depends on the intended application. Constitutive promoters can be used to produce continuous transcription of the reporter gene. Inducible promoters can be used to provide external control for varying the levels of transcription. To mimic the transcription of some endogenous gene, one can use the identical promoter of the endogenous gene to be imaged. This allows the use of reporter genes to indirectly monitor the expression of an endogenous gene. Two reporter gene approaches are discussed: the herpes simplex type 1 virus thymidine kinase (HSVI-tk) and the dopamine type 2 receptor (D2R). Other approaches have also been developed and are reviewed elsewhere.

Similarly, the emission detection of an intracellularly trapped radionuclide can be used as a reporter to monitor the expression of genes of interest or therapeutic genes (e.g., tumor necrosis factor (TNF)) or to monitor therapeutic gene delivery to targeted angiogenic endothelium of tumor blood vessel. For example, a vector used to transfect a target cell line can include TNF and a microbial hydrogenase encoding nucleic acid sequence both under the control of a tissue specific promoter (e.g., breast cancer specific promoter, prostate cancer specific promoter) other promoter. Propagation of the TNF encoding vector in a target tissue or cell line can be monitored by treating the target cell line with a radionuclide and detecting emissions from the intracellularly trapped radionuclide following enzymatic reduction by concomitantly expressed microbial hydrogenase.

### EXAMPLE III

### Development of a Novel Reporter-Probe System for Enzymatic Reduction and Intracellular Trapping of Technetium ⁹⁹m-Pertechnetate Using Microbial Technetium Reductases and SPECT Imaging

Several reporter genes currently available include Aequorea-derived fluorescent proteins (AFPs), luciferases, and herpes simplex virus type I thymidine kinase (HSV1-tk) genes. These reporter genes have limited clinical applications because of poor tissue penetration and resolution (optical and bioluminescent) or non-specific background activity of endogenous enzyme (Thymidine Kinase) and the requirement for an expensive radio-labeled probe for imaging (HSV1-tk).

Tc99m O4—is widely used as a radiopharmaceutical in nuclear medicine because of its maging properties, low toxicity, low cost, and because it is readily available. Enzymatic reduction and intracellular trapping of Tc99m O4—has been reported in bacteria, including Escherichia coli (E. coli), sulfate-reducing bacteria Desulfovibrio desulfuricans (DD) and in eukaryotic microorganisms, including human parasite Trichomonas vaginalis (TV). In E. coli, the Tc(VII) reductase activity has been identified as hydrogenase 3 encoded by the HycE and HycG genes in the hyc operon. Two genes, HydA and HydB encode for the hydrogenase activity in DD and TV, which is expressed as a heterodimer. Fe(III) and radioactive isotopes of Uranium and Technetium are reduced in a reduction reaction catalyzed by an oxygen tolerant hydrogenase.

This Example shows that microbial technetium reductases are able to mediate enzymatic reduction and intracellular trapping of Tc99m O4—in bacterial and mammalian cells, when this microbial enzyme is ectopically expressed. Upon reduction of ^{99m}Tc pertechnatate, the metal significantly reduces its solubility falling out of solution and binding to proteins. Inducible bacterial and constitutive mammalian expression of D. desulfuricans hydrogenase allows the insoluble compound to become closely associated with the cells and can then be used to detect the cells through gamma radiation detectors and SPECT imaging.

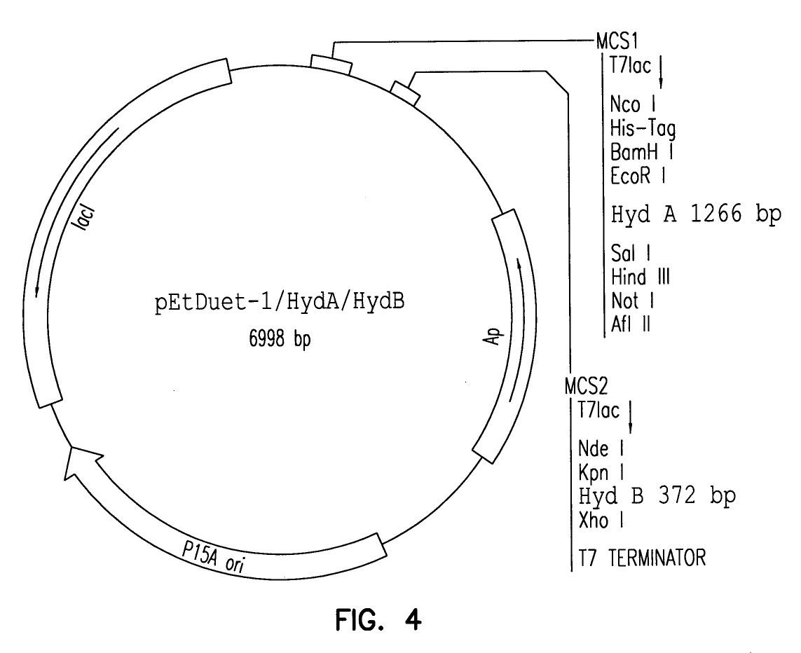

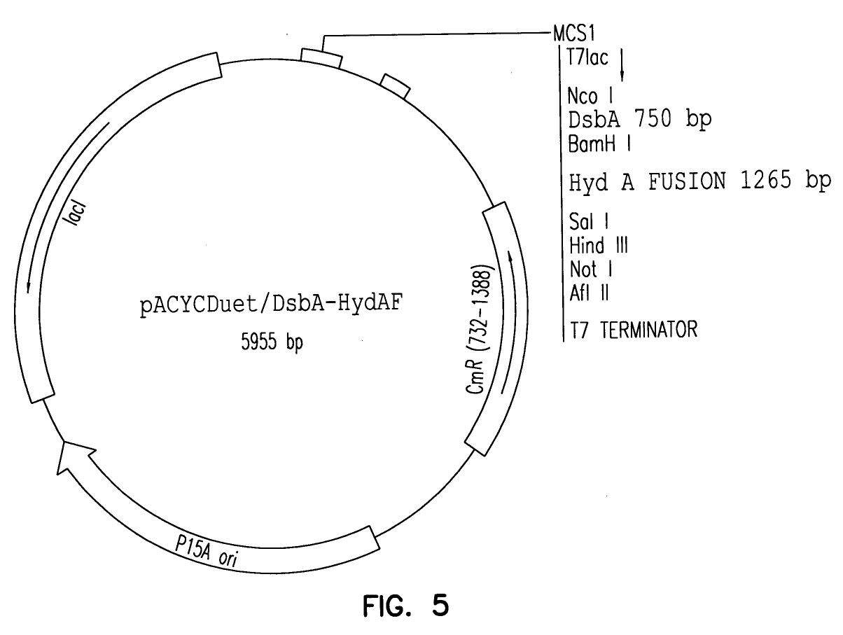

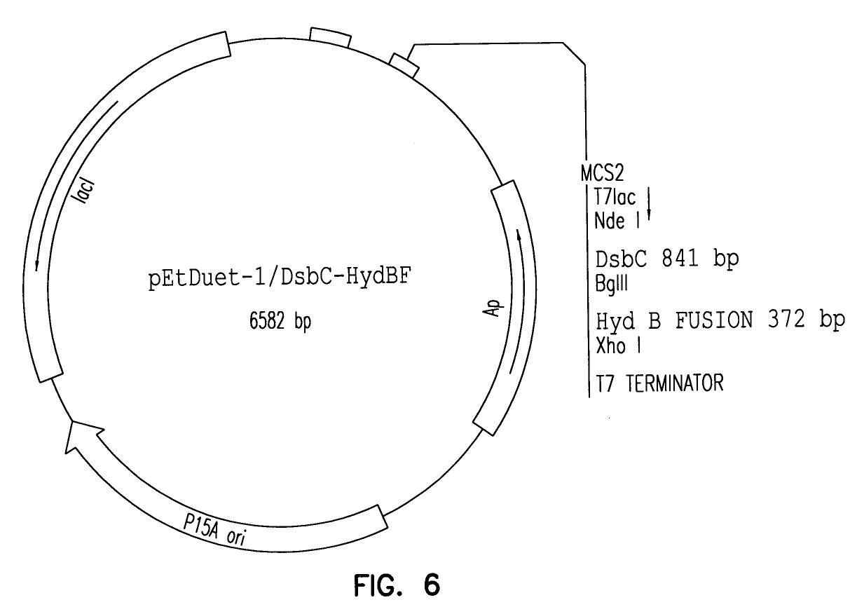

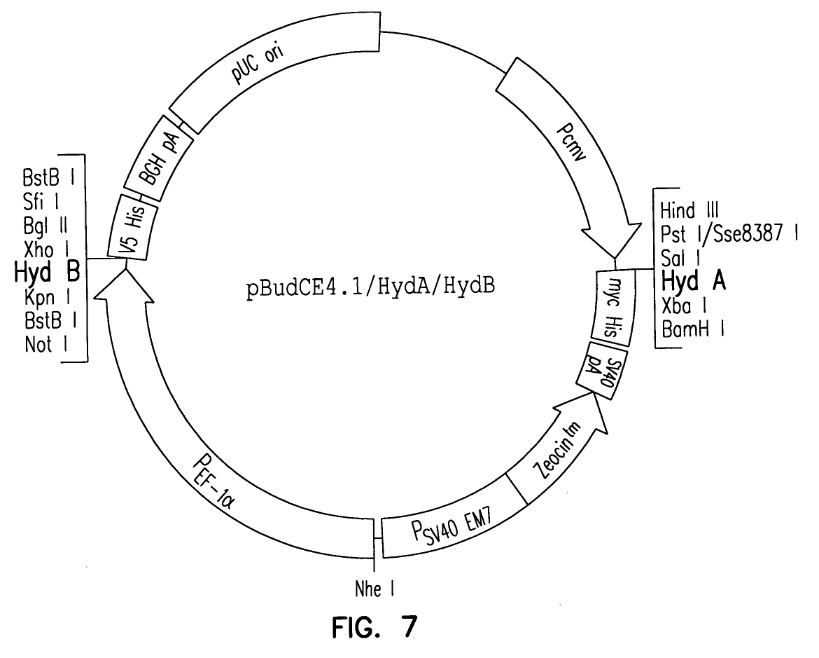

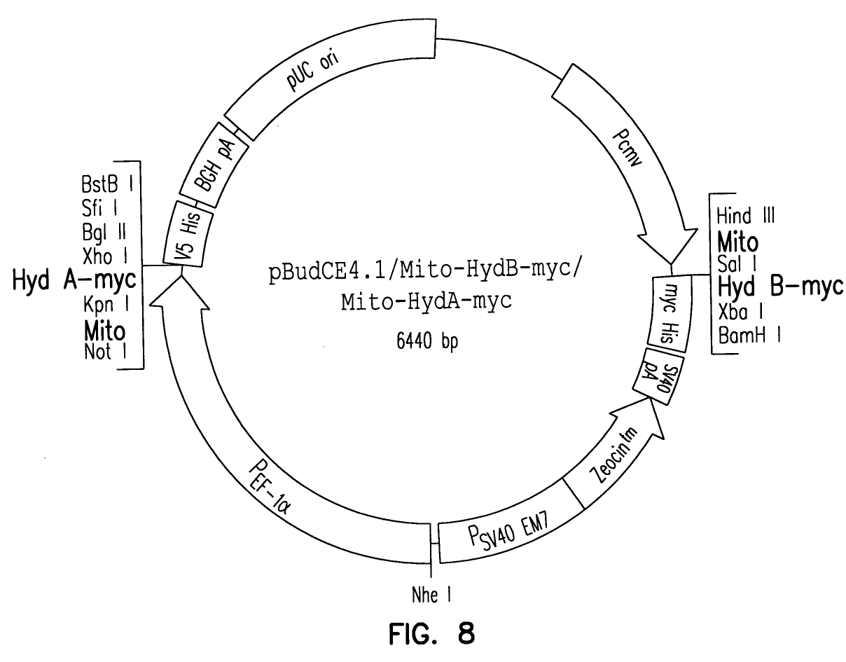

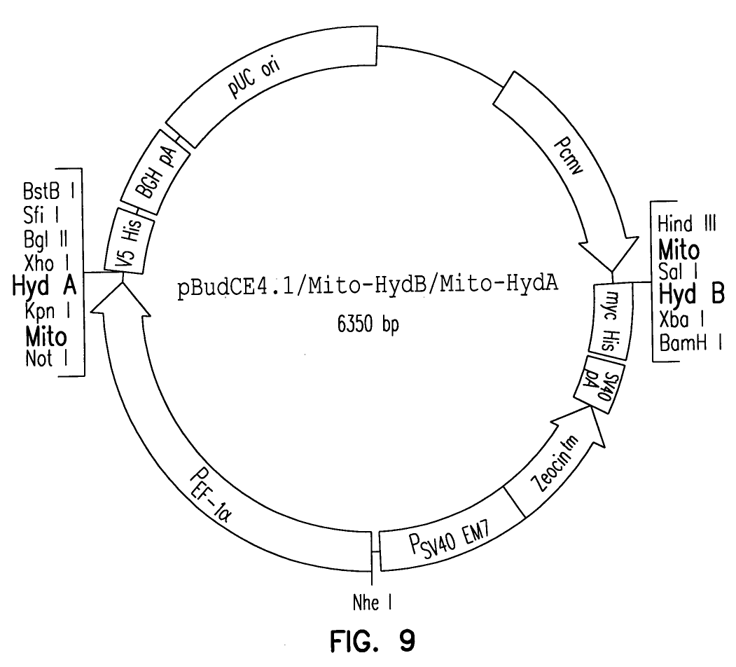

A novel reporter gene-probe system for molecular imaging was developed to investigate the use of microbial reductases for enzymatic reduction and intracellular trapping of Technetium 99m pertechnetate in the bacteria and animal cells. The hydrogenase genes from the organisms D. sulfuricans, T. vaginalis and E. coli (MC4100 strain) were subcloned and overexpressed in bacterial and mammalian vectors (see FIGS. 4-9). Bacteria overexpressing the D. sulfuricans HydA and HydB genes showed significantly higher trapping of Tc99m O4—in vitro as compared to control cells. In the E. coli expression system, periplasmic directing domains DsbA and DsbC were ligated in fusion to Hyd A and Hyd B respectively, resulting in increased 99mTc association with the bacteria. Because hydrogenase routinely interacts with cytochrome c in bacteria, mammalian vectors were constructed using mitochondrial targeting sequences, so that this interaction might facilitate a higher activity in eukaryotic cells.

Bacterial expression vectors were designed following standard molecular biology techniques. D. desulfuricans hydrogenase genes, Hyd A and Hyd B were PCR amplified using primers incorporated with unique restriction sites at the ends. The genes were inserted into pET-Duet1 (Novagen) vector under T7 RNA polymerase driven promoter through sequential restriction digestion and ligation. Periplasmic signal sequences were extracted from vectors pET-39b and pET-40b (Novagen) and ligated in-frame with start codon deficient Hydrogenase genes. Mammalian expression vectors were designed with mitochondrial signal sequences from pCMV/myc/mito, fused in frame by PCR amplification and ligation with both HydA and HydB genes. Additionally, the myc epitope was added in-frame at the C-terminus to facilitate subcellular localization and immunoprecipations.

E. coli BL21(DE3) Star (Invitrogen) was transformed with various constructs and grown on the appropriate selective media agars. In case of periplasmic fusion hydrogenases, dual co-transformation was accomplished using antibiotic selection markers, ampicillin and chloramphenicol. Transformed cells were grown at 37° C. in a standard laboratory shaker under aerobic conditions to an optical density at 600 nm (O.D.₆₀₀) of 0.6 and then induced with 1 mM IPTG under a standard T7 promoter induction protocol. One ml aliquots of cells were collected at 0, 1, 2, and 3 hour time points following induction and mRNA was extracted using a bacterial mRNA extraction mini kit (Qiagen). 100 ng of the mRNA samples was DNAse treated (Invitrogen) according to the manufacturer's protocol. RNA quality was then analyzed using an Agilent 2100 Bioanalyzer, prokaryotic total RNA nano-chip. RT-PCR reactions were performed using the internal specific primers for HydA, Forward: 5′-ACTGTCACCATTGGCGATGC (SEQ ID NO:8); Reverse: 5′-TGCCTTGCTGGACGACATTC (SEQ ID NO:9) and Hyd B, Forward: 5′-AGTATATGACCGACCGCATA (SEQ ID NO:10), Reverse 5′-TTCGTAAGGATACCCGGTTGC (SEQ ID NO:11) and one step RT-PCR kit (Qiagen).

Tc99m O4—reduction assays were performed as follows: Bacterial cultures were grown to an O.D.₆₀₀ of 0.6 in a simple rotary shaker under aerobic conditions. HydA and HydB genes were induced with 1 mM IPTG in the presence of 15 mCi of 99mTc Pertechnetate and ten ml aliquots of bacterial cultures were collected at 0, 1, 2, and 3 hr time points. The cells were spun down at 3500 rpm for 15 min, washed twice with 5 ml phosphate buffered saline (PBS) and the supernatant was collected and saved for later analysis. Control bacteria transformed with Duet vectors only were treated as controls. Total counts in the total cell pellet, 100 μl of media supernatant, and 1 ml of each wash, were then assayed using a gamma counter. Ten microliters of 99mTc pertechnetate was assayed to provide internal control between experiments.

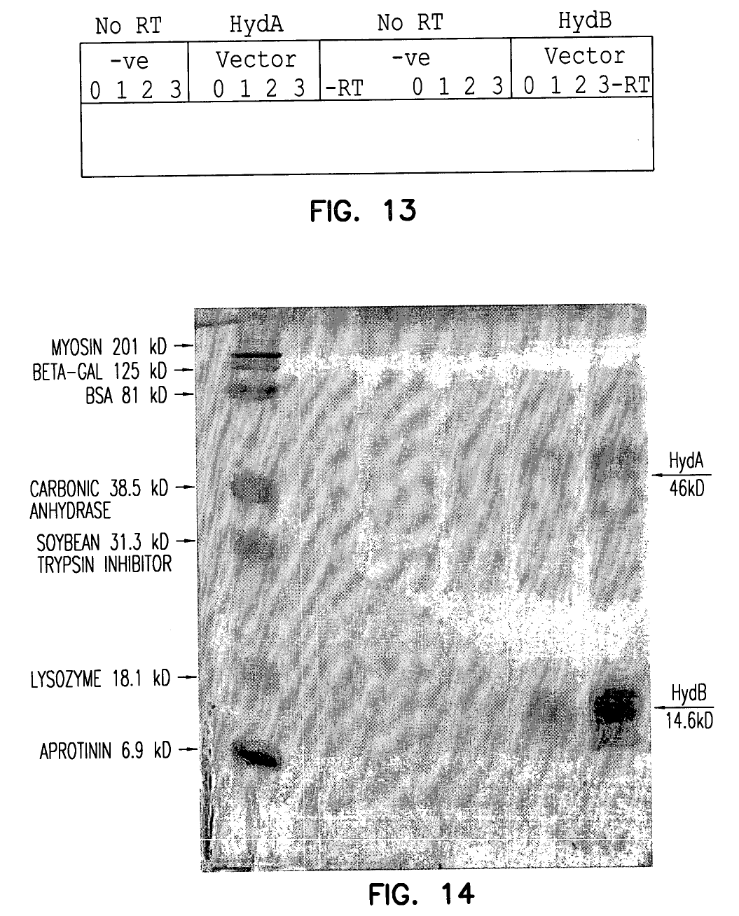

Induction of both Hydrogenase and Dsb-Hyd fusion proteins by IPTG resulted in marked up-regulation of their associated mRNA transcripts as a function of time over a three-hour induction period, as visualized by RT-PCR with hydrogenase specific primers (FIG. 13). Analysis of cell lysates by SDS polyacrylamide gel electrophoresis confirmed that the Hydrogenase proteins were highly expressed (FIG. 14).

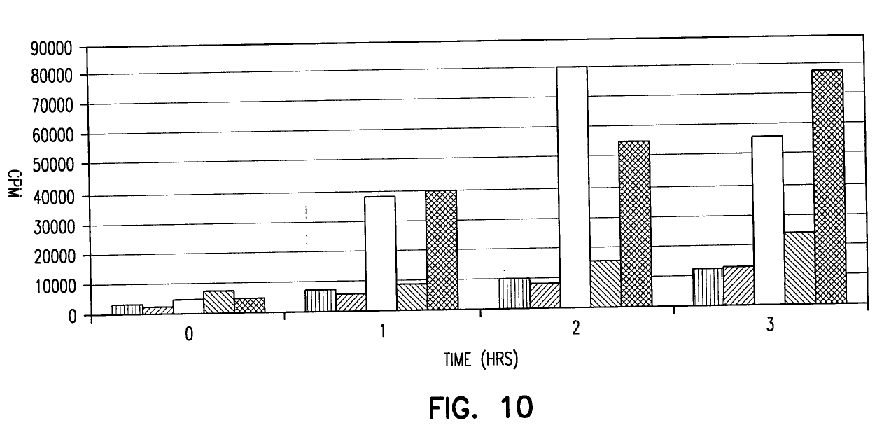

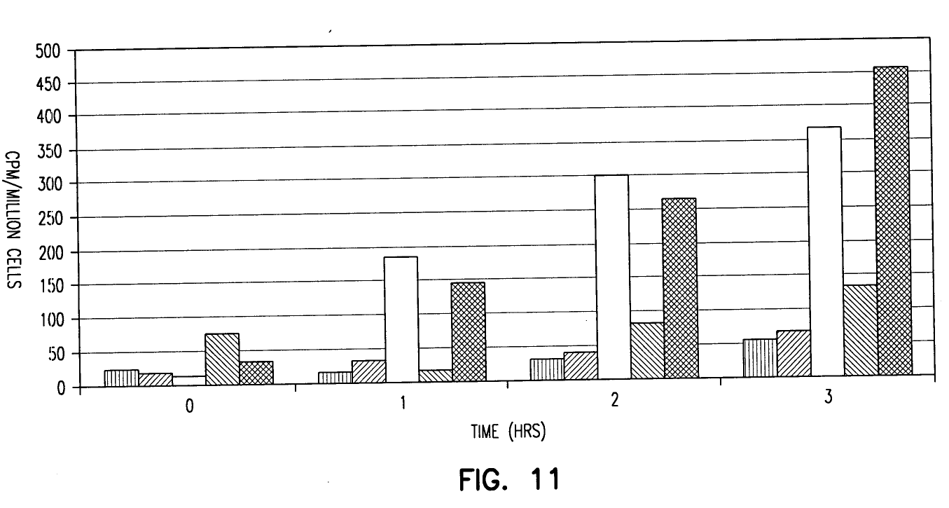

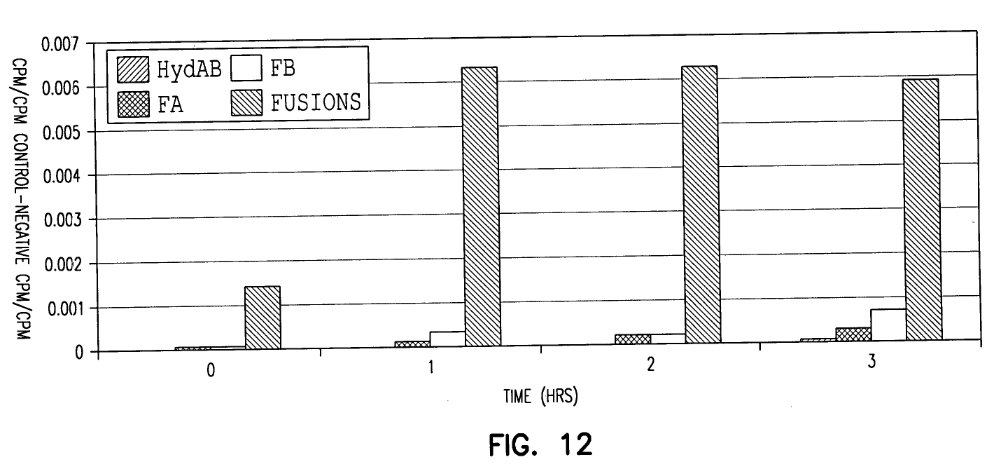

As shown in FIGS. 10 and 11, Hydrogenase activity increased over several hours after IPTG induction, as measured by the amount of radioactivity retained in the pellet after rinsing away residual soluble technetium. In FIG. 12, the amount of radioactivity recorded in counts per minute was divided by the total number of counts expected per 10 ml of culture. The experimental values were determined by subtracting the control bacterial normalized CPM from the normalized CPM from the hydrogenase vector(s) transformed bacteria. The values obtained were therefore normalized counts per minute for the different cell lines.

Periplasmic fusion vectors containing DsbA-HydA and DsbC-HydB were separately induced in the presence of 99mTc to determine if one part of the heterodimeric enzyme was sufficient for completing the reduction. There was a noticeable increase in radioactivity within cells containing the pETDuet1/HydA/HydB vector relative to control cells with the pET-Duet+PAcYCDuet vector. However, this increase was negligible when compared to cells containing both HydA and HydB in separate (individual) Dsb fusion vector constructs. Surprisingly, entrapment of radionuclide by cells containing the Hyd B-Dsb fusion alone was higher than that of both fusion hydrogenase products together (FIGS. 10-12, Table 4).

TABLE 4
Counts Per Minute for Control (pET-Duet + pACYCDuet, column 2),
pETDuet/HydA/HydB (column 3), pACYC-DsbA-HydA +
pETDuet/DsbC-HydB (column 4), pACYC-DsbA-HydA alone (column 5)
and pETDuet/DsbC-HydB alone (column 6).

| TIME (HR) | D + P | HYDA/B | DSB FUSIONS | FA | FB |
|---|---|---|---|---|---|
| 0 | 2777 | 2074 | 4185 | 7074 | 4643 |
| 1 | 6939 | 5543 | 38129 | 8503 | 40064 |
| 2 | 10045 | 8031 | 80021 | 15323 | 55201 |
| 3 | 11944 | 12339 | 56611 | 23935 | 77462 |

Therefore, a novel reporter-probe system was developed for imaging using Tc99m O4-. The results illustrated herein indicate that D. desulfuricans Hydrogenase genes can be induced strongly in E. coli using the well established T7 lac induction system and that this up-regulation results in active enzyme under aerobic conditions. Because Hydrogenase is a periplasmic enzyme in D. desulfuricans, there is a significant increase in the reducing activity of this enzyme induced in the periplasm of E. coli.

While both parts of the heterodimer of the hydrogenase do show individual activity in the periplasmic fusions, the HydB fusion consistently showed higher reduction activity versus HydA Fusion.

In future, for mammalian cells, In vitro assays for the reduction of ⁹⁹mTc will be performed on cell lines transiently and stably transfected with eukaryotic vector constructs. These vectors contain strong constitutive promoters, which will allow the viability of hydrogenase activity in eukaryotic cells to be tested extensively. Because hydrogenase interacts with Cytochrome C in D. desulfuricans, for significant eukaryotic activity to be observed, mitochondrial targeting vectors will be investigated as a possible mechanism for obtaining maximal reductive activity.

Subsequent work will utilize a mouse models of bacterial infection followed by oral administration of IPTG to induce the hydrogenases and subsequent tail vein injection of ^{99m}Tc Pertechnetate to verify that simple blood flow will allow the local delivery, reduction of ^{99m}Tc and in vivo imaging.

Cancer cell lines stably transfected with the reporter genes will be utilized for mouse xenograft tumor models and will provide an opportunity for in vivo studies of ^{99m}Tc trapping and SPECT imaging.

### REFERENCES

Lloyd J R, Cole J A, Macaskie L E. Reduction and removal of heptavalent technetium from solution by Escherichia coli. J. Bacteriol. 1997; 179:2014-2021.

Lloyd J R, Nolting H F, Sole V A, Bosecker K, Macaskie L E. Technetium reduction and precipitation by sulfphate-reducing bacteria. Geomicrobiol. 1998; 15:43-56.

Lloyd J R, Ridley J, Khizniak T, Lyalikova N N, Macaskie L E. Reduction of technetium by Desulfovibrio desulfuricans: biocatalyst characterization and use in a flow-through bioreactor. Appl Environ Microbiol. 1999; 65:2691-2696.

Having now fully described this invention, it will be appreciated by those skilled in the art that the same can be performed within a wide range of equivalent parameters, concentrations, and conditions with due experimention without departing from the spirit and scope of the invention.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method for intracellular radionuclide trapping comprising:
a) obtaining a mammalian target cell that was transfected with a vector comprising a nucleic acid segment encoding a microbial hydrogenase and a promotor operatively linked to the microbial hydrogenase encoding nucleic acid segment; and
b) diffusing a radionuclide into the mammalian target cell, wherein the radionuclide is reduced intracellularly by the microbial hydrogenase expressed by the mammalian target cell to produce a trapped radionuclide.

2. The method of claim 1, further comprising contacting the mammalian target cell with a reducing agent.

3. The method of claim 1, wherein the microbial hydrogenase is Escherichia coli—Hydrogenase 3, Desulfovibrio desulfuricans—Hydrogenase or Trichomonas vaginalis—Hydrogenase.

4. The method of claim 1, wherein the nucleic acid segment encoding the microbial hydrogenase comprises a nucleic acid sequence with any one of SEQ ID NO:1-7 or a nucleic acid sequence with at least 90% homology to any one of SEQ ID NO:1-7.

5. The method of claim 1, wherein the radionuclide is Technitium-99m, Technitium-94m, Rhenium 186, Rhenium 188 or a combination thereof.

6. The method of claim 1, wherein the mammalian target cell is human.

7. The method of claim 1, wherein the mammalian target cell is a human cancer cell.

8. The method of claim 1, further comprising detecting an emission from the trapped radionuclide.

9. The method of claim 1, wherein the detecting is by positron emission topography or by single photon emission computerized tomography.

10. The method of claim 1, wherein the radionuclide emits a β-particle.

11. The method of claim 1, wherein the radionuclide emits a γ-particle.

12. The method of claim 1, wherein an emission from the trapped radionuclide is cytotoxic to the mammalian target cell.

13. The method of claim 1, wherein the vector further comprises a second nucleic acid segment encoding a transgene.

14. A method for reporting transgene expression comprising:
a) diffusing a radionuclide into a mammalian target cell, wherein the radionuclide is trapped intracellularly by hydrogenase-mediated reduction of the radionuclide by a microbial hydrogenase expressed by the mammalian target cell; wherein the mammalian target cell was transfected with a vector that includes a nucleic acid segment encoding a microbial hydrogenase operatively linked to a first promoter, and a nucleic acid segment encoding a transgene operatively linked to a second promoter, and the first promoter and the second promoter allow an RNA polymerase to transcribe the microbial hydrogenase and the transgene; and
b) detecting emission from the trapped radionuclide.

15. The method of claim 14, further comprising diffusing a reducing agent into the mammalian target cell.

16. The method of claim 14, wherein the first promoter and the second promoter are identical.

17. The method of claim 14, wherein the microbial hydrogenase is Escherichia coli—Hydrogenase 3, Desulfovibrio desulfuricans—Hydrogenase or Trichomonas vaginalis—Hydrogenase.

18. The method of claim 14, wherein the nucleic acid segment encoding the microbial hydrogenase comprises a nucleic acid sequence with any one of SEQ ID NO:1-7 or a nucleic acid sequence with at least 90% homology to any one of SEQ ID NO:1-7.

19. The method of claim 14, wherein the radionuclide is Technitium-99m, Technitium-94m, Rhenium 186, Rhenium 188 or a combination thereof.

20. The method of claim 14, wherein the mammalian target cell is human.

21. The method of claim 14, wherein the mammalian target cell is a human cancer cell.

22. The method of claim 14, wherein the radionuclide emits a β-particle.

23. The method of claim 14, wherein the radionuclide emits a γ-particle.

24. The method of claim 14, wherein an emission from the trapped radionuclide is cytotoxic to the mammalian target cell.

25. The method of claim 14, wherein the detecting is by positron emission topography or by single photon emission computerized tomography.

26. A method for intracellular radionuclide trapping comprising diffusing Technitium-99m into a mammalian target cell that was transfected with a vector which includes a nucleic acid segment encoding T. vaginalis—hydrogenase operatively linked to a promoter, wherein the Technitium-99m is trapped intracellularly by hydrogenase-mediated reduction of the Technitium-99m by a T. vaginalis hydrogenase expressed by the mammalian target cell to produce a trapped radionuclide.

27. The method according to claim 26, further comprising diffusing a reducing agent into the mammalian target cell.

28. A mammalian cell comprising:
a) a vector that includes a nucleic acid segment encoding a microbial hydrogenase and a promoter operatively linked thereto; and
b) an intracellularly trapped radionuclide; wherein the radionuclide has been reduced in a reaction catalyzed by a microbial hydrogenase expressed from the vector.

29. The mammalian cell of claim 28, wherein the nucleic acid segment encoding the microbial hydrogenase comprises a nucleic acid sequence with any one of SEQ ID NO:1-7 or a nucleic acid sequence with at least 90% homology to any one of SEQ ID NO:1-7.

30. The mammalian cell of claim 28, wherein the microbial hydrogenase is Escherichia coli—Hydrogenase 3, Desulfovibrio desulfuricans—Hydrogenase or Trichomonas vaginalis—Hydrogenase.

31. The mammalian cell of claim 28, wherein the radionuclide is Technitium-99m, Technitium-94m, Rhenium 186, Rhenium 188 or a combination thereof.

32. The mammalian cell of claim 28, wherein said mammalian target cell is a human cancer cell.

33. A mammalian cell complex comprising a mammalian cell that contains a radionuclide, wherein the mammalian cell was transfected with a vector that contains a nucleic acid segment encoding a microbial hydrogenase and a promoter operatively linked to the nucleic acid segment, and wherein the microbial hydrogenase catalyzes reduction of the radionuclide.

34. A mammalian cell comprising:
a) a vector comprising a first nucleic acid segment encoding a microbial hydrogenase operatively linked to a first promoter and a second nucleic acid segment encoding a transgene operatively linked to a second promoter; and
b) an radionuclide intracellularly trapped by reduction in an enzyme-mediated reaction with the expressed microbial hydrogenase.

35. The mammalian cell of claim 34, wherein the nucleic acid segment encoding the microbial hydrogenase comprises a nucleic acid sequence with any one of SEQ ID NO:1-7 or a nucleic acid sequence with at least 90% homology to any one of SEQ ID NO:1-7.

36. The mammalian cell of claim 34, wherein the microbial hydrogenase is Escherichia coli—Hydrogenase 3, Desulfovibrio desulfuricans—Hydrogenase or Trichomonas vaginalis—Hydrogenase.

37. The mammalian cell of claim 34, wherein the radionuclide is Technitium-99m, Technitium-94m, Rhenium 186, Rhenium 188 or a combination thereof.

38. The mammalian cell of claim 34, wherein the first and second promoters are the same promoter.

39. The mammalian cell of claim 34, further comprising a gene product encoded by the transgene and a transgenically expressed catalytically active microbial hydrogenase.

40. A kit comprising:
a) a mammalian cell having a transgenic vector with a nucleic acid segment encoding a microbial hydrogenase that is catalystically active in the mammalian cell, the nucleic acid segment operatively linked to a first promoter, and wherein the vector includes at least one restriction endonuclease site suitable for cloning a transgene which is linked operatively to a second promoter, and
b) a radionuclide.

41. A kit comprising:
a) a transgenic vector with a nucleic acid segment encoding a microbial hydrogenase, the nucleic acid segment operatively linked to a first promoter, and wherein the vector includes at least one restriction endonuclease site suitable for cloning a transgene which is linked operatively to a second promoter, and
b) a radionuclide.

42. The kit of claim 41, wherein the nucleic acid segment encoding the microbial hydrogenase comprises a nucleic acid sequence with any one of SEQ ID NO:1-7 or a nucleic acid sequence with at least 90% homology to any one of SEQ ID NO:1-7.

43. The kit of claim 41, wherein the microbial hydrogenase is Escherichia coli—Hydrogenase 3, Desulfovibrio desulfuricans—Hydrogenase or Trichomonas vaginalis—Hydrogenase.

44. The kit of claim 41, wherein the radionuclide is Technitium-99m, Technitium-94m, Rhenium 186, Rhenium 188 or a combination thereof.

45. The kit of claim 41, wherein the first and second promoters are the same promoter.
